(19) United States
(12) Patent Application Publication
(10) Pub. No.: US 2021/0285974 A1
(43) Pub. Date: Sep. 16, 2021

(54) RAPID POLYMERIZATION OF POLYPHENOLS

(71) Applicant: UNIVERSITY OF WASHINGTON, Seattle, WA (US)

(72) Inventors: Xiaohu GAO, Seattle, WA (US); Junwei LI, Seattle, WA (US)

(21) Appl. No.: 16/341,273

(22) PCT Filed: Oct. 30, 2017
(86) PCT No.: PCT/US2017/059131

§ 371 Date: Apr. 11, 2019

Related U.S. Application Data
(60) Provisional application No. 62/414,117, filed on Oct. 28, 2016.
(60) Provisional application No. 62/504,995, filed on May 11, 2017.

(51) Int. Cl. G01N 33/94; G01N 33/535

(52) U.S. Cl. CPC G01N 33/9413; G01N 33/535; G01N 2333/4737; G01N 2333/96455; G01N 2333/5751; G01N 2333/72; G01N 2333/475

(57) ABSTRACT

This disclosure provides a method for polymerizing polyphenols to provide polyphenol polymers using peroxidase and similar catalysis. In various aspects, it provides a method for polymerizing a polyphenol (e.g., polydopamine or a derivative or conjugate thereof) on a surface comprising polymerizing the polyphenol, a method for detecting an analyte comprising polymerizing a polyphenol, and an assay kit comprising a polyphenol (e.g., dopamine or a dopamine derivative). In one embodiment, a method for polymerizing a polyphenol includes contacting the polyphenol and an oxidant with an enzyme having peroxidase-like activity, under conditions sufficient to polymerize the polyphenol. In another embodiment, a method for depositing a polyphenol polymer (e.g., a polydopamine) includes providing, at a target site, an enzyme having peroxidase-like activity immobilized at the surface; and polymerizing, at the target site, a polyphenol in the presence of an oxidant and the enzyme to provide the polyphenol polymer, deposited on the surface.

### CROSS-REFERENCE TO RELATED APPLICATIONS

This application claims the benefit of priority of U.S. Provisional Patent Application no. 62/414,117, filed Oct. 28, 2016, and U.S. Provisional Patent Application No. 62/504,995, filed May 11, 2017, each of which is hereby incorporated herein by reference in its entirety.

### STATEMENT OF GOVERNMENT SUPPORT

This invention was made with government support under Grant No. R21 CA192985, awarded by the National Institutes of Health. The government has certain rights in the invention.

### BACKGROUND OF THE DISCLOSURE

### Field of the Disclosure

This disclosure relates generally to a method for polymerizing polyphenols, such as dopamine and its derivatives. In certain embodiments, the present disclosure relates to a method for depositing a polyphenol polymer (e.g., polydopamine) on a surface by polymerizing a polyphenol (e.g., dopamine or a dopamine derivative), to a method for detecting an analyte by polymerizing a polyphenol (e.g., dopamine or a dopamine derivative), and to an assay kit comprising a polyphenol (e.g., dopamine or a dopamine derivative).

### Technical Background

As recent advances in medicine rapidly unravel the genomic and proteomic signatures of disease development, progression, and response to therapy, sensitive and quantitative analysis of disease biomarkers (e.g., DNA, RNA, and proteins) has become increasingly important in the era of precision medicine where diagnostic and therapeutic decisions are tailored towards individual patients. In parallel, to address the challenge in sensitive and multiplexed biomarker analysis, a large variety of exquisitely designed imaging and detection technologies have also been developed in the past decade. These enabling technologies, often leveraging the unique properties of colloidal nanostructures (e.g., quantum dots, magnetic nanoparticles, and plasmonic nanoparticles) and precisely engineered sensor devices (e.g., nanowire sensors, cantilevers, and microfluidic channels) are so sensitive that their detection limits are commonly seen at the single-molecule level, where low-abundance targets such as circulating oligonucleotides, proteins, viruses, and cells can be enumerated with polymerase chain reaction (PCR)-like sensitivity. Despite these remarkable achievements in biotechnology laboratories, broad adoption of these technological innovations by biological and clinical laboratories, and consequently, the impact thereof, has been limited. Resistance to adoption stems from multiple factors, including complex protocols and specialized reagents and equipment. Moreover, these technologies require new infrastructure, which increases up-front adoption costs, and reduces persistent output and cross-laboratory cross-platform consistency.

Accordingly, there remains a need for high-sensitivity detection methods that avoid specialized reagents or equipment, and/or can be performed with minimal alteration to existing laboratory infrastructure.

### SUMMARY OF THE DISCLOSURE

One aspect of the disclosure is a method for polymerizing a polyphenol, including:
providing a polyphenol;
providing an enzyme having peroxidase-like activity;
contacting the polyphenol and an oxidant with the enzyme having peroxidase-like activity, under conditions sufficient to polymerize the polyphenol to form a polyphenol polymer.

Another aspect of the disclosure is a method for depositing a polyphenol polymer on a surface, the method including
providing, at a target site, an enzyme having peroxidase-like activity immobilized at the surface; and
polymerizing, at the target site, a polyphenol in the presence of an oxidant and the enzyme to provide the polyphenol polymer, deposited on the surface.

Another aspect of the disclosure is a method for detecting an analyte, the method including
providing a sample comprising the analyte; and a primary detection reagent, linked to an enzyme having peroxidase-like activity;
incubating the sample in the presence of the primary detection reagent to provide a target site comprising a complex of the analyte and the detection reagent;
polymerizing, at the target site, a polyphenol in the presence of an oxidant and the enzyme to provide a polyphenol polymer; and
detecting the presence of polyphenol polymer.

Another aspect of the disclosure is an assay kit, including
an intermediate detection reagent, capable of binding an analyte;
a primary detection reagent linked to an enzyme having peroxidase-like activity, the primary detection reagent capable of binding the intermediate detection reagent; and
a polyphenol (e.g., dopamine or a dopamine derivative).

Other aspects of the disclosure will be evident from the disclosure herein.

### BRIEF DESCRIPTION OF THE DRAWINGS

FIG. 1 is a schematic illustration of certain embodiments of the methods of the disclosure (EASE). Dopamine (colorless) slowly oxidizes in the presence of air (O₂ as oxidant) and produces brown-black polydopamine (PDA). This polymerization process can be sped up by approximately 300 times under horseradish peroxidase (HRP) catalysis (H₂O₂ as oxidant). See Example 1, below.

FIG. 2 is A) an image of dopamine polymerization under conventional and HRP-catalyzed conditions at various time points; and B) a graph of the extinction measured at 700 nm for the samples shown in (A). See Example 1, below.

FIG. 3 is a plot of the normalized extinction spectra of polydopamine and dopamine, as discussed in more detail in Example 1, below.

FIG. 4 is a schematic illustration of HRP-catalyzed PDA deposition on a solid support. When protein density on the solid support is low (for example only HRP is present), the majority of the FDA molecules diffuse away. For solid supports (e.g., flat surface and membrane) with high protein density (e.g., in cells and surfaces blocked with protein molecules for reduced nonspecific binding), rapid and localized deposition of PDA occurs due to the reactivity of PDA to nearby amines (rich in proteins) and other reactive groups, leading to formation of a dark spot. See Example 1, below.

FIG. 5 is a set of images showing membranes immobilized with bovine serum albumin (BSA) alone, HRP alone, or HRP/BSA, before and after exposure to dopamine, as discussed in more detail in Example 1, below. Scale bar, 5 mm.

FIG. 6 is a schematic illustration of immunohistochemistry (IHC) performed according to certain embodiments of the methods of the disclosure. Cells are labeled with an intermediate detection reagent (1′Ab) and a primary detection reagent (2′Ab-HRP complex) sequentially, and exposed to dopamine. Localized FDA deposition (dark brown) indicates the spatial and abundance information of the analyte. See Example 2, below.

FIG. 7 is a set of bright-field images of cells stained via IHC, performed according to certain embodiments of the methods of the disclosure, with different magnifications showing cytoplasmic and nuclear staining of HSP90 and Lamin A, respectively, as discussed in more detail in Example 2, below. Scale bar, 50 μm.

FIG. 8 is a bright-field image of a large population of HSP90 cells stained via IHC, performed according to certain embodiments of the methods of the disclosure, showing specific cytoplasmic localization of HSP90. See Example 2. Scale bar, 200 μm.

FIG. 9 is a bright-field image of a large population of Lamin A cells stained via IHC, performed according to certain embodiments of the methods of the disclosure, showing specific nuclear localization of Lamin A. See Example 2. Scale bar, 200 μm.

FIG. 10 is a set of images comparing the staining patterns of HSP90 and Lamin A before and after quantum dot (QD) absorption. The top panels are bright-field micrographs of conventional IHC cell staining (DAB, 3,3′diaminobenidene as the substrate). The bottom panels are fluorescence micrographs of conventional immunofluorescence (IF) cell staining using QD-labeled 2′Ab (positive control). Scale bar, 100 μm. See Example 2,

FIG. 11 is a set of bright-field images of HSP90 stained according to certain embodiments of the methods of the disclosure, showing increased specificity relative to negative controls. Mismatched anti-mouse ((M)-HRP), an absence of primary detection reagent (2′Ab-HRP), or an absence of dopamine produces negligible signals. See Example 2. Scale bar, 100 μm.

FIG. 12 is a graph of the quantitative staining intensities of the samples of Example 2. Statistical analysis of cells in four random field-of-views shows significant differences between the experiment and control groups. ***P<0.001 by two-tailed t-test, error bars indicating s.d.

FIG. 13 is a bright-field image of a large population of cells stained according to certain embodiments of the methods of the disclosure, while using an isotype 1′Ab as the control intermediate detection reagent (rabbit IgG). Negligible signals were observed. See Example 2. Scale bar, 200 μm.

FIG. 14 is a graph of the quantitative staining stabilities, upon storage, of the samples of Example 2. Error bars, s.d. over four different images.

FIG. 15 is a set of bright-field images of a cell sample of Example 2, imaged periodically over ˜100 days. Stains, stored in 1× PBS at 4° C., showed no decay over time. Scale bar, 200 μm.

FIG. 16 includes a schematic illustration of cells stained via IHC, performed according to certain embodiments of the methods of the disclosure (IHO-EASE), and further labeled with amine-functionalized quantum dots (QD-PEG-NH₂; QD-NH₂); and a comparison of a fluorescence micrograph image of QD-NH₂-labeled HSP90 cells (bottom right) with the bright-field image of the cells before QD-NH₂-labeling (bottom left). See Example 2.

FIG. 17 is a set of fluorescence micrographs of cells stained via IHC, performed according to certain embodiments of the methods of the disclosure, and various controls (lacking intermediate detection reagent and/or dopamine), as discussed in more detail in Example 2, below. Scale bar, 50 μm.

FIG. 18 is a graph of the quantitative fluorescence intensities of the samples shown in FIG. 17. See Example 2. The intensity difference between the experiment and controls are highly significant. ***P<0.001 by two-tailed t-test. Error bars, s.d. over four different images.

FIG. 19 is a fluorescence micrograph showing HSP-90 cells (88 pM 1′Ab) stained under various conditions, as discussed in more detail in Example 2 below: experimental group (left panels) and control group using isotype rabbit IgG as the intermediate detection reagent (1′Ab) (right panels), using either an embodiment of the methods of the disclosure (EASE; top panels) or conventional IF (bottom panels). Scale bar, 100 μm; exposure time, 100 ms. To better illustrate the background levels, long exposure (2 second) images were also shown for the control panels.

FIG. 20 is a graph showing the quantitative fluorescence intensities of the experimental and control samples shown in FIG. 19, as discussed in more detail in Example 2, below. Comparison of the controls for each (using an isotype intermediate detection reagent) showed no significant background increase. P>0.1, not significant by two-tailed t-test. Error bars, s.d. over four different images.

FIG. 21 is a graph showing the quantitative improvement in IF staining intensity provided by certain embodiments of the methods of the disclosure (EASE). See Example 2. Signal intensity obtained through certain embodiments of the methods of the disclosure at 88 pM intermediate detection reagent (1′Ab) is roughly the same as the intensity obtained with conventional IF at 11 nM 1′Ab. Error bar, s.d. over four different images.

FIG. 22 is a set of false-color (heat map) fluorescence images of cells stained with various concentrations of intermediate detection reagent (1′Ab), as discussed in more detail in Example 2, below. Scale bar, 100 μm.

FIG. 23 is a set of fluorescence images of four analytes (HSP90, Lamin A, Ki-67, and Cox-4) stained according to certain embodiments of the methods of the disclosure (EASE), or according to conventional methods, at an intermediate detection reagent (1′Ab) dilution of 1:25,000. See Example 2. Scale bar, 50 μm.

FIG. 24 is a graph showing the quantitative fluorescence intensities of the samples of FIG. 23, as discussed in more detail in Example 2, below. The differences are statistically significant. ***P<0.001 by two-tailed t-test. Error bars, s.d. over four different images.

FIG. 25 is a set of fluorescence images of GAPDH stained by IF performed according to certain embodiments of the methods of the disclosure (EASE) and conventional IF before RNAi, as discussed in more detail in Example 2, below. Scale bar, 100 μm.

FIG. 26 is a set of fluorescence images of GAPDH stained according to certain embodiments of the methods of the disclosure (EASE) and GAPDH stained via conventional IF 36 hours and 60 hours post-RNAi, as discussed in more detail in Example 2, below. Despite the majority of GAPDH being degraded, the trace remainder is still detectible by certain embodiments of the methods of the disclosure, but not by conventional IF. Scale bar, 100 μm.

FIG. 27 is a schematic illustration of a suspension microarray assay performed according to certain embodiments of the methods of the disclosure (EASE). Fluorescent microspheres coated with Abs (IgG) (model capture reagents) capture and immobilize 2′Ab-biotin (a model analyte) in solution. The analyte molecule is detected by FDA deposition catalyzed by streptavidin (SA)-HRP complex (a model primary detection reagent) followed by QD-NH₂ adsorption. See Example 3, below.

FIG. 28 is a set of images showing the effect of PDA coating on microsphere fluorescence (1×109 beads 12 nM 2′Ab-biotin), as discussed in more detail in Example 3, below. The dark microsphere suspension shows successful FDA deposition, while the microscopy images show no obvious fluorescence change before and after the deposition. Scale bar, 5 μm.

FIG. 29 is a graph showing the fluoresce spectra of green fluorescence beads before (broken line) and after FDA coating (EASE process), as discussed in more detail in Example 3, below. The two samples contained the same concentration of beads.

FIG. 30 is a set of representative fluorescence images of the microspheres of Example 3, and the corresponding quantitative flow cytometry data, showing strong QD fluorescence signals only when both QD-PEG-NH₂ and dopamine were present (1×10₆ beads ml₋₁, 12 pM 2′Ab-biotin). Scale bar, 3 μm. Error bars, s.d. over three replicates.

FIG. 31 is a set of quantitative flow cytometry histograms showing that QDs bind onto the bead surfaces of Example 3 only when dopamine is polymerized on the microsphere surface and amine-functionalized QDs are used. The left panels show the fluorescence from the dye-doped microsphere, and the right panels show QD fluorescence.

FIG. 32 is a set of representative fluorescence images of single-bead samples of Example 3, and corresponding quantitative flow cytometry data (1×10₆beads ml₋₁), showing a 100-fold improvement in detection sensitivity (12 pM to 1.2 fM) from a conventional suspension microarray to a suspension microarray performed according to certain embodiments of the methods of the disclosure (EASE). Scale bar, 3 μm.

FIG. 33 is a graph showing verification of the specificity of the microarray of Example 3. At an analyte (biotinylated 2′Ab) concentration of 12 pM, certain embodiments of the methods of the disclosure (EASE) can increase sensitivity relative to concentration suspension microarrays, to easily detect an analyte (blank bars), When the analyte is missing (control, dashed bars), the background signal intensity of the assays are indistinguishable (P>0.1, NS, not significant by two-tailed t-test). Error bars, s.d. over three replicates.

FIG. 34 is a set of images showing fluorescence detection of mouse IgG (capture reagent), immobilized on green microspheres, and rabbit IgG (capture reagent), immobilized on yellow microspheres, when biotinylated anti-mouse IgG and anti-rabbit IgG were used as analytes, in combination with amine-functionalized QDs, as discussed in more detail in Example 3, below. Mismatched antibody pairs did not produce QD fluorescence. Sale bar, 3 μm.

FIG. 35 is a set of images showing two-color microsphere mixtures, prepared according to Example 3, incubated with only one analyte, anti-rabbit IgG. QD deposition only occurred on the yellow microspheres (having rabbit IgG immobilized on the surface thereof). Scale bar, 15 μm.

FIG. 36 is a graph of single-bead counting of the samples of Example 3, showing detection of the anti-rabbit IgG at 100% accuracy (100 beads of each color were counted),

FIG. 37 is a schematic illustration of ELISA performed according to certain embodiments of the methods of the disclosure (EASE). A layer of PDA is coated around the target complex, which allows a large number of HRP polypeptides to adsorb. These HRP polypeptides, in turn, catalyze conversion of the substrate (e.g., TMB) at a significantly enhanced rate, See Example 4, below.

FIG. 38 is an image showing the detection sensitivity of ELISA performed according to certain embodiments of the methods of the disclosure (EASE), using mouse IgG as a model analyte in comparison with conventional ELISA, as discussed in more detail in Example 4, below. Colored solutions are visualized in EASE wells at analyte concentrations as low as 10₋₁₃ g ml₋₁, while the conventional assay only produces detectable colors at 10₋₈ to 10₋₉ g ml₋₁ concentration range.

FIG. 39 is a set of graphs comparing the quantitative sensitivities of ELISA performed according to certain embodiments of the methods of the disclosure (EASE) and conventional ELISA over the full analyte concentration range (left) and over a range close to the assays' limits-of-detection (LODs) (right). See Example 4. Improvements of approximately 3 orders of magnitude were observed. Error bars, s.d. over three replicates.

FIG. 40 is a graph showing verification of the specificity of ELISA performed according to certain embodiments of the methods of the disclosure (EASE). At an analyte (mouse IgG) concentration of 154 pg ml⁻¹, the analyte presence can be detected by ELISA performed according to certain embodiments of the methods of the disclosure, but not by conventional ELISA. Without the analyte molecule, the background signal intensity of the assays are indistinguishable (P>0.1, NS, not significant by two-tailed t-test). Error bars, s.d. over three replicates.

FIG. 41 is a graph showing confirmation of the specificity and cross-reactivity of the assay of Example 4. At the analyte (HIV p24) concentration of 60 fg ml⁻¹, the analyte presence can be detected by the assay of Example 4, with very low background from the controls (without analyte molecule). To further test the selectivity, 1,000× concentrated proteins (60 pg m1⁻¹) including human serum albumin (HSA), HTLV-1 p24, and SIV p27 were spiked into 1× (60 fg ml⁻¹) HIV p24 solution, and probed by ELISA performed according to certain embodiments of the methods of the disclosure. No significant cross-reactivity was observed for HSA. The non-specific proteins (HTLV-1 p24 and SIV p27) that are more similar to p24 only produced appreciable signals at 1000X concentrations relative to p24.

FIG. 42 is a set of graphs comparing the quantitative sensitivities of ELISA performed according to certain embodiments of the methods of the disclosure (EASE) and conventional ELISA for four analytes, HIV p24, KLK3, CRP, and VEGF. See Example 4. Error bars, s.d. over three replicates.

FIG. 43 is a graph showing the average of LOD improvements for all four analytes shown in FIG. 42, as discussed in more detail in Example 4. The improvement for each analyte was about 1,200-fold.

FIG. 44 is an image of the lateral flow strip of Example 4. Each cassette contains three strips. Capture reagents (antibodies) are immobilized along the test line of each strip, as discussed in more detail in Example 4, below.

FIG. 45 is an image of the HIV p24 strip tests of Example 4, with or without an embodiment of the methods of the disclosure (EASE). Positive signals (indicated by the arrow) were observed at 10 ng ml⁻¹ and 10 pg ml⁻¹ for the experimental strips, but the conventional strips could only detect as low as 10 ng ml⁻¹. Each strip represented three replicates.

FIG. 46 is an image verifying the specificity of the lateral flow tests of Example 4. Control experiments were the analyte (p24 antigen) is absent showed no detectable signals, with or without an embodiment of the methods of the disclosure.

FIG. 47 is a graph of the LOD values (obtained from 9 runs performed on different days) of the HIV p24 assay and control of Example 5. The average LOD of ELISA performed according to certain embodiments of the methods of the disclosure (EASE) is 2.84 fg ml⁻¹, 1,060-fold lower than that of conventional ELISA.

FIG. 48 is a set of graphs comparing the quantitative sensitivities of ELISA performed according to certain embodiments of the methods of the disclosure (EASE) and conventional ELISA for HIV p24. See Example 5. Error bars, s.d. over three replicates.

FIG. 49 is a set of graphs comparing the first date at which HIV infenction became detectable via the ELISA performed according to certain embodiments of the methods of the disclosure (EASE) and conventional ELISA, as discussed in more detail in Example 5. Positive detection was made within the first week for ELISA performed according to certain embodiments of the methods of the disclosure and FOR, whereas conventional ELISA detected infection only 2-3 later, when the viral load was high.

FIG. 50 is a schematic illustration of the cerebral cortex (CTX) of a mouse brain (Gregma: −2.79 mm). See Example 6.

FIG. 51 is a representative fluorescence image of CRFR1 neurons in a mouse CTX, stained according to certain embodiments of the methods of the disclosure, counter stained with DAPI, as discussed in more detail in Example 6, below. Scale bar, top panels, 200 μm. Scale bar, middle panel, 100 μm. Scale bar, bottom panels, 5 μm. A large number of CRFR1-positive cells are observed through IF, performed according to certain embodiments of the disclosure (EASE), but not with conventional IF (see FIG. 52). Interneurons (I) and pyramidal neurons (II) are indicated by arrows. Apical dendrites of pyramidal neurons are shown by the arrows in composite image (II).

FIG. 52 is a representative fluorescence image of conventionally stained CRFR1 neurons in a mouse CTX, counter stained with DAPI, as discussed in more detail in Example 6, below. Scale bar, top panels, 200 μm. Scale bar, bottom panel, 100 μm.

FIG. 53 is a representative control fluorescence image of CRFR1 neurons in a mouse CTX, stained according to certain embodiments of the methods of the disclosure, but without an intermediate detection reagent (1′Ab) (Ease/Control), as discussed in more detail in Example 6, below. Scale bar, top panels, 200 μm. Scale bar, middle panel, 100 μm. Scale bar, bottom panels, 5 μm.

FIG. 54 is a set of representative fluorescent images of ZIKV in placental chorionic villi (nuclei counter-stained with DAPI), stained according to Example 7, below. Scale bar, 100 μm. ZIKV infected cells indicated by arrows can only be observed through IF performed according to certain embodiments of the methods of the disclosure (EASE), but not with conventional IF. Staining specificity is verified using controls (without intermediate detection reagent (1′Ab), or non-infected placentas). Dashed lines, cytotrophoblast cell layer (identified by morphology). Infected cells appear within the chorionic villus core and villi beneath in close proximity to the cytotrophoblast cell layer, as indicated by the arrows. The red background signal is due to tissue autofluorescence, which can be reduced under confocal imaging where the excitation source is a laser (narrow band).

FIG. 55 is a set of representative confocal fluorescence images showing the distribution of ZIKV in tissue sections (left panel) and single cells (right panel), as discussed in more detail in Example 7, below. Brighter signals indicate ZIKV, and darker signals indicate DAPI. Dashed lines, cytotrophoblast cell layer (identified by morphology). Infected cells appear within the chorionic villus core and villi beneath in close proximity to the cytrophoblast cell layer. Scale bar, 50 μm.

FIG. 56 is a set of representative fluorescence micrographs of PD-L1 expression in pancreatic specimens from the patient (SU-09-21157), samples counter-stained with DAPI. Scale bar, 100 μm. Brighter signals indicate PD-L1, and darker signals indicate DAPI. PD-L1 staining can be easily observed through IF performed according to certain embodiments of the methods of the disclosure (EASE), but very difficult using the conventional IF. The control experiment (without intermediate detection reagent (l′Ab)) did not show detectable signals.

FIG. 57 is a set of representative fluorescence micrographs of PD-L1 expression in pancreatic specimens from the patient (SI-10-26808), samples counter-stained with DAPI. Scale bar, 100 μm. Brighter signals indicate PD-L1, and darker signals indicate DAPI. PD-L1 staining can only be observed through IF performed according to certain embodiments of the methods of the disclosure (EASE), but not conventional IF. The control experiment (without intermediate detection reagent (I′Ab)) did not show detectable signals.

### DETAILED DESCRIPTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

In various aspects and embodiments, the disclosure relates to a method for polymerizing a polyphenol, including providing a polyphenol, providing an enzyme having a peroxidase-like activity, contacting the polyphenol and an oxidant with the enzyme having peroxidase-like activity, under conditions sufficient to polymerize the polyphenol. The present inventors have determined that enzymes with peroxidase-like activity (such as peroxidases, phosphatases, and ribozymes) can greatly speed the rate of polymerization of polyphenols such as dopamine, providing polyphenol polymers such as polydopamine at fast rates.

As described in detail below, this discovery allows for the targeted deposition of polyphenol polymers at a surface. But, in other embodiments, the polymerization can be performed using aqueous solution-phase chemistry to provide polyphenol polymer. In many embodiments, the polyphenol polymer will precipitate from aqueous solution to form a solid polymer, which can be collected for use in a separate process, or can be allowed to deposit on a surface in contact with the aqueous solution (e.g., in a non-targeted manner). Accordingly, the methods described herein can be used to form surface coatings of polyphenol polymers on a variety of surfaces, or to form polymer that is collected and used in a further process. The person of ordinary skill in the art will determine appropriate reaction conditions based on the disclosure herein. For example, in certain embodiments as otherwise described herein, the polyphenol is present in the reaction mixture in a concentration in the range of 1-100 mg/mL, e.g., 1-75 mg/mL, 1-50 mg/mL, 1-25 mg/mL, 5-100 mg/mL, 5-75 mg/mL, 5-50 mg/mL, 5-25 mg/mL, 10-100 mg/mL, 10-75 mg/mL or 10-50 mg/mL. In certain embodiments as otherwise described herein, the oxidant is present in the reaction mixture in an amount in the range of 0.005-2 M, e.g., in the range of 0.005-1 M, or 0.005-0.5 M, or 0.005-0.1 M, or 0.01-2 M, or 0.01-1M, or 0.01-0.5 M, or 0.01-0.1 M. The reaction can be conducted at a variety of pH values, e.g., in the range of 1-11, or 4-11, or 7-11, or 7-9.

In one aspect, the disclosure relates to a method for depositing a polyphenol polymer (e.g., polydopamine) on a surface. The method includes providing, at a target site, an enzyme having peroxidase-like activity immobilized at the surface, and polymerizing, at the target site, a polyphenol (e.g., dopamine or a dopamine derivative) in the presence of an oxidant and the enzyme to provide the polyphenol polymer, deposited on the surface. The disclosure demonstrates that such a method provides for rapid deposition of commonly available materials onto a surface.

While the examples described below focus on the use of dopamine and derivatives thereof (including conjugates thereof), based on the present disclosure the person of ordinary skill in the art will understand that the methods described herein can be used to polymerize a variety of polyphenols. As used herein, a “polyphenol” is a compound having a polyhydroxyphenyl moiety, e.g., a dihydroxyphenyl moiety or a trihydroxyphenyl moiety (e.g., as a substituent or fused as part of a ring system). Examples of polyphenols include dopamine and dopamine derivatives as described below. Other examples of polyphenols include elegeic acid, theaflavin-3-gallage, gallic acid, tannic acid, pyrogallol, catechol, catechin, epigallocatechin, epigallocatechin, quercetin, morin, naringenin, rutin, naringin, phloroglucinol, hydroquinone, resorcinol, hydroxyhydroquinone, resveratrol, as well as derivatives of these materials (such as conjugates thereof). The person of ordinary skill in the art will appreciate that derivatives of polyhydroxyphenyl-bearing compounds can include any modified that is capable of polymerizing to provide a polyphenol polymer. The methods can be used, for example, with extracts of materials such as green tea, black tea, cacoa bean, and red wine. In certain embodiments, the polyphenol has a molecular weight of no more than 1000 g/mol, e.g., no more than 800 g/mol, or even no more than 500 g/mol. As used herein, a polyphenol polymer is a polymer of a polyphenol, e.g., a homopolymer of a single polyphenol or a copolymer of a plurality of different polyphenols.

In certain embodiments of the disclosure, the polyphenol is dopamine or a derivative thereof. As described in more detail below, a polyphenol polymer formed by polymerization of dopamine or a derivate thereof (i.e., a “polydopamine”) can have a high optical density at certain wavelengths, which can advantageously allow for optical detection of the degree of polymerization. As used herein, the term “dopamine derivative” includes covalently modified dopamine (e.g., ortho or meta to the aminoethyl group), and dopamine otherwise conjugated to a chemical moiety (e.g., a fluorescent tag, biotin, etc.). The person of ordinary skill in the art will appreciate that the dopamine derivatives of the methods described herein may be any modified dopamine compound that is capable of polymerizing to provide a polydopamine.

For example, in certain embodiments, the polyphenol has the structure A or B below

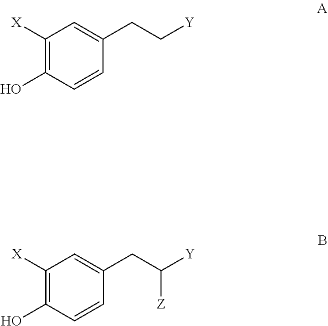

in which X is OH, O(C₁-C₄ alkyl), (C₁-C₄ alkyl), preferably OH; Y is NH2, biotin, PEG-linked biotin, or a fluorophore moiety; and Z is COOH, NH2, biotin, PEG-linked biotin, or a fluorophore moiety.

As used herein, the term “polydopamine” refers to a polymer of dopamine or a dopamine derivative, e.g., a homopolymer of polydopamine or a derivative thereof, or a copolymer of a plurality of polyphenols including polydopamine or a derivative thereof. The person of ordinary skill in the art will appreciate that the term “polydopamine” includes the polymerization product of dopamine or a dopamine derivative provided by the methods described herein.

As described above, in one aspect of the methods of the disclosure, the deposition method includes providing, at a target site, an enzyme having peroxidase-like activity immobilized at a surface. In certain embodiments of the methods as otherwise described herein, the enzyme is adsorbed onto the surface. For example, in certain embodiments of the methods as otherwise described herein, the enzyme is absorbed onto a membrane, e.g., a nitrocellulose membrane. In certain embodiments of the methods as otherwise described herein, the enzyme is linked to the surface via a streptavidin-biotin interaction. In certain embodiments of the methods as otherwise described herein, the enzyme is linked to the surface via an antibody-antigen interaction. In certain embodiments of the methods as otherwise described herein, the enzyme is linked to the surface via a silane coupling agent. For example, in certain embodiments of the methods as otherwise described herein, the enzyme is linked to a silica surface via a trialkoxysilane moiety.

Of course, as described above, other embodiments provide polymerization methods in which the enzyme having peroxidase-like activity is not immobilized at a surface. For example, in various embodiments, the enzyme having peroxidase-like activity is in aqueous solution or suspension when it is contacted with the polyphenol and the oxidant.

Another aspect of the disclosure is method for detecting an analyte. In various aspects and embodiments, the disclosure demonstrates the method to be compatible with virtually all common biodetection and bioimaging techniques (see, e.g., Table 16, below), and capable of providing sensitivities that are orders of magnitude higher than those conventional techniques. The method includes providing a sample comprising the analyte and a primary detection reagent, linked to an enzyme having peroxidase-like activity, and incubating the sample in the presence of the primary detection reagent to provide a target site comprising a complex of the analyte and the detection reagent. The method also includes polymerizing, at the target site, a polyphenol (e.g, dopamine or a dopamine derivative) in the presence of an oxidant and the enzyme to provide a polyphenol polymer (e.g., polydopamine), and detecting the presence of the polyphenol polymer (e.g., the polydopamine). In various aspects and embodiments, certain embodiments of the methods as otherwise described herein are referred to as enzyme-accelerated signal enhancement (EASE).

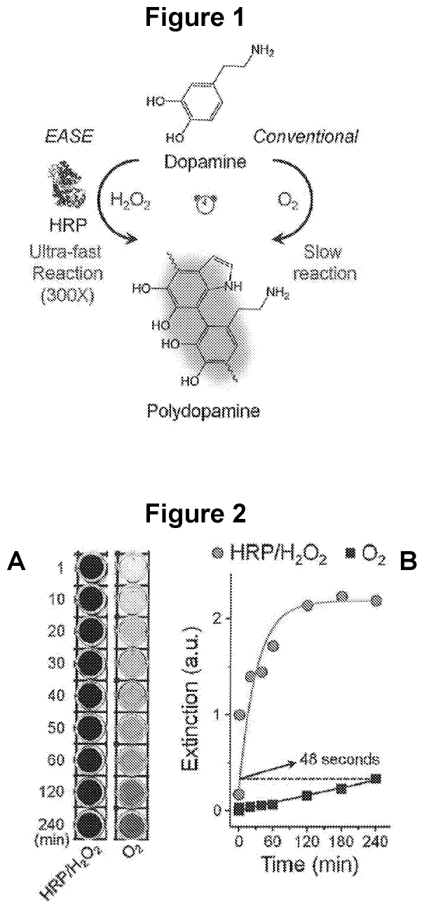

The person of ordinary skill in the art will appreciate that polyphenol polymers such as polydopamines are versatile coating materials in a variety of surface treatment fields. For example, self-adherent polydopamine films have been shown to form spontaneously, but slowly, on a wide range of surfaces using a dip-coating protocol. Advantageously, the present inventors have determined that the rate of polymerization of polyphenols such as dopamine and dopamine derivatives is increased by a factor of hundreds in the presence of an enzyme having peroxidase-like activity (e.g., horseradish peroxidase (HRP): see FIG. 1). Thus, the polymerization methods described herein can be used to provide desirable surface coatings of polyphenol polymer much more quickly than in conventional methods. The present inventors have further determined that, by taking advantage of peroxidase-like-activity-catalyzed deposition, polyphenol polymers such as polydopamines may be deposited in a site-specific manner and subsequently detected, according to various aspects and embodiments of the methods described herein.

As described above, in one aspect of the methods of the disclosure, the detection method includes providing a primary detection reagent, linked to an enzyme having peroxidase-like activity. In certain embodiments of the methods as otherwise described herein, the primary detection reagent comprises an antibody. For example, in certain embodiments of the methods as otherwise described herein, the primary detection reagent comprises a monoclonal antibody, e.g., a monoclonal antibody to another antibody, to a human immunodeficiency virus (HIV) antigen (such as, for example, p24), a corticotrophin releasing factor (CRF) receptor, a Zika virus (ZIKV) antigen, or an immune regulatory antigen (such as, for example, PD-L1). In certain embodiments of the methods as otherwise described herein, the primary detection reagent comprises streptdavidin. In certain embodiments, the primary detection reagent comprises a peptide, an oligonucleotide, or a derivative thereof (e.g., biotin-labeled deriviatives).

In certain embodiments of the methods as otherwise described herein, the primary detection reagent is capable of binding the analyte. In other embodiments of the methods as otherwise described herein, the detection method further comprises providing an intermediate detection reagent capable of binding the analyte. In certain such embodiments, the detection reagent is capable of binding the intermediate detection reagent, and incubation is further in the presence of the intermediate detection reagent, to provide a target site comprising a complex of the analyte, intermediate detection reagent, and primary detection reagent. For example, in certain embodiments of the methods as otherwise described herein, the intermediate detection reagent comprises an antibody. For example, in certain embodiments of the methods as otherwise described herein, the intermediate detection reagent comprises a monoclonal antibody, e.g., a monoclonal antibody to a human immunodeficiency virus (HIV) antigen (such as, for example, p24), a corticotrophin releasing factor (CRF) receptor (such as, for example, CRFR1), a Zika virus (ZIKV) antigen, or an immune regulatory antigen (such as, for example, PD-L1). In certain embodiments of the methods as otherwise described herein, the primary detection reagent comprises a monoclonal antibody, e.g., a monoclonal antibody to another antibody, to a prostate-specific antigen (kallikrein-3 (KLK3)), to a c-reactive protein (CRP), to a vascular endothelial growth factor (VEGF), to a human immunodeficiency virus (HIV) antigen (such as, for example, p24), a corticotrophin releasing factor (CRF) receptor, a zika virus (ZIKV) antigen, or an immune regulatory antigen (such as, for example, PD-L1). In certain embodiments of the methods as otherwise described herein, the intermediate detection reagent comprises a biotin-labeled affinity molecule.

As described above, in one aspect of the methods of the disclosure, the method includes providing a sample comprising the analyte. In certain embodiments of the methods as otherwise described herein, the analyte is immobilized on a cell surface, or localized in a cell compartment (e.g., an immunohistochemistry or immunofluorescence analyte, e.g., Lamin A or heat shock protein (HSP)-90). In certain embodiments of the methods as otherwise described herein, the analyte is bound to a capture reagent, the capture reagent immobilized on a solid support (e.g., a sandwich-assay analyte, e.g., an enzyme-linked immunosorbent assay (ELISA) analyte, e.g., KLK3, CRP, VEGF, p24, CRFR1, a ZIKV antigen, or PD-L1) In certain such embodiments, the capture reagent comprises an antibody, e.g., a monoclonal antibody. In certain such embodiments, the solid support comprises a microsphere.

As described above, in one aspect of the methods of the disclosure, the method includes detecting the presence of the polyphenol polymer (e.g., polydopamine). In certain embodiments of the methods as otherwise described herein, detection comprises measuring the absorption or emission of the polyphenol polymer (e.g., polydopamine). For example, in certain embodiments of the methods as otherwise described herein, measuring the absorption or emission of the polyphenol polymer (e.g., polydopamine) comprises observing the color change of a target site caused by the absorption of the polyphenol polymer (e.g., polydopamine) after polymerization. In another example, in certain embodiments of the methods as otherwise described herein, measuring the absorption or emission of the polyphenol polymer (e.g., polydopamine) comprises quantitatively measuring the emission of the polyphenol polymer (e.g., polydopamine) polymerized from a polyphenol comprising a fluorescent tag (e.g., dopamine conjugated to a fluorescent tag).

In certain embodiments of the methods as otherwise described herein, the detection method further comprises incubating the polydopamine in the presence of a secondary detection reagent. For example, in certain embodiments of the methods as otherwise described herein, the secondary detection reagent comprises an enzyme capable of catalyzing the conversion of a chromogenic substrate (e.g., HRP and enzyme conjugates HRP-streptavidin and streptavidin-poly HRP). In certain such embodiments, detection comprises measuring the absorption or emission of the chromogenic substrate (e.g., 3,3′,5,5′-tetramethylbenzidine (TMB), 3,3′-diaminobenzidine (DAB), or 2,2′-azino-bis(3-ethylbenzothiazoline-6-sulphonic acid) (ABTS)). Advantageously, the present inventors have determined that reactivity of a polydopamine towards the amine, sulfhydryl, and phenol groups of polypeptides allows for localization at the target site of a high concentration of the enzyme capable of catalyzing the conversion of a chromogenic substrate. In another example, in certain embodiments of the methods as otherwise described herein, the secondary detection reagent comprises an amine-functionalized tag. Similarly advantageously, the present inventors have determined that reactivity of a polyphenol polymer (e.g., polydopamine) towards amine groups allows for localization at the target site of a high concentration of the amine-functionalized tag. In certain such embodiments, the amine-functionalized tag comprises a quantum dot. In other such embodiments, the amine-functionalized tag comprises an amine-functionalized dye (e.g., a fluorescent dye, e.g., cyanine 3 (Cy3)). In certain such embodiments, detection comprises measuring the absorption or emission of the secondary detection reagent.

In certain embodiments of the methods as otherwise described herein, the detection method comprises providing a sample comprising the analyte, the analyte immobilized on a cell surface or localized in a cell compartment, an intermediate detection reagent (e.g., a monoclonal antibody) capable of binding the analyte, and a primary detection reagent (e.g., a monoclonal antibody) linked to an enzyme having peroxidase-like activity (e.g., HRP). In certain such embodiments, the method further includes incubating the sample in the presence of the intermediate detection reagent, to provide a target site comprising a complex of the analyte and intermediate detection reagent. In certain such embodiments, the method further includes incubating the sample in the presence of the primary detection reagent, to provide a target site comprising a complex of the analyte, the intermediate detection reagent, and the primary detection reagent. In certain such embodiments, the method further includes polymerizing, at the target site, a polyphenol (e.g., dopamine or a dopamine derivative) in the presence of an oxidant (e.g., H₂O₂) and the enzyme to provide a polyphenol polymer (e.g., polydopamine), and detecting the presence of the polyphenol polymer. In certain such embodiments, detection comprises measuring the absorption or emission of a polyphenol polymer (e.g., polydopamine). In other such embodiments, the method further includes incubating the polyphenol polymer (e.g., polydopamine) in the presence of a secondary detection reagent (e.g., an amine-functionalized tag, e.g., an amine-functionalized quantum dot). In certain such embodiments, detection comprises measuring the absorbance or emission of the secondary detection reagent.

In certain embodiments of the methods as otherwise described herein, the detection method comprises providing a sample comprising the analyte (e.g., an analyte comprising biotin), the analyte bound to a capture reagent (e.g., a monoclonal antibody), the capture reagent immobilized on a microsphere, and a primary detection reagent (e.g., streptavidin) linked to an enzyme having peroxidase-like activity (e.g., HRP). In certain such embodiments, the method further includes incubating the sample in the presence of the primary detection reagent, to provide a target site comprising a complex of the analyte and the primary detection reagent. In certain such embodiments, the method further includes polymerizing, at the target site, a polyphenol (e.g., dopamine or a dopamine derivative) in the presence of an oxidant (e.g., H₂O₂) and the enzyme to provide a polyphenol polymer (e.g., polydopamine), and detecting the presence of the polyphenol polymer. In certain such embodiments, the method further includes incubating the polyphenol polymer (e.g., polydopamine)in the presence of a secondary detection reagent (e.g., an amine-functionalized tag, e.g., an amine-functionalized quantum dot). In certain such embodiments, detection comprises measuring the absorption or emission of the secondary detection reagent.

In certain embodiments of the methods as otherwise described herein, the detection method comprises providing a sample comprising the analyte, the analyte bound to a capture reagent (e.g., a monoclonal antibody), the capture reagent immobilized on a solid support, and a primary detection reagent (e.g., a monoclonal antibody) linked to an enzyme having peroxidase-like activity (e.g., HRP). In certain such embodiments, the primary detection reagent is capable of binding the analyte. In certain such embodiments, the method further includes incubating the sample in the presence of the primary detection reagent, to provide a target site comprising a complex of the analyte and the primary detection reagent. In certain such embodiments, the method further includes polymerizing, at the target site, a polyphenol (e.g., dopamine or a dopamine derivative) in the presence of an oxidant (e.g., H₂O₂) and the enzyme to provide a polydopamine, and detecting the presence of polydopamine. In certain such embodiments, the method further includes incubating the polyphenol polymer (e.g., polydopamine) in the presence of a secondary detection agent comprising an enzyme capable of catalyzing the conversion of a chromogenic substrate (e.g., HRP). In certain such embodiments, detection comprises measuring the absorption or emission of the chromogenic substrate (e.g., DAB).

As described above, in one aspect of the methods of the disclosure, the method includes providing a sample comprising the analyte. In certain embodiments of the methods as otherwise described herein, the analyte is a Lamin antigen, e.g., Lamin A. In certain embodiments of the methods as otherwise described herein, the analyte is a heat shock protein (HSP), e.g., HSP-90. In certain embodiments of the methods as otherwise described herein, the analyte is a kallikrein 3 (KLK3) antigen. In certain embodiments of the methods as otherwise described herein, the analyte is a C-reactive protein (CRP). In certain embodiments of the methods as otherwise described herein, the analyte is a vascular endothelial growth factor (VEGF) antigen. In certain embodiments of the methods as otherwise described herein, the analyte is a human immunodeficiency virus (HIV) antigen, e.g., p24. In certain embodiments of the methods as otherwise described herein, the analyte is a corticotrophin releasing factor (CRF) receptor, e.g., CRFR1. In certain embodiments of the methods as otherwise described herein, the analyte is a zika virus (ZIKV) antigen. In certain embodiments of the methods as otherwise described herein, the analyte is an immune regulator antigen, e.g., programmed death-ligand 1 (PD-L1).

As described above, the present inventors have determined that the various aspects and embodiments of the methods described herein are compatible with virtually all common biodetection and bioimaging techniques. For example, in certain embodiments of the methods as otherwise described herein, the sample comprising an analyte bound to a capture reagent, the capture reagent immobilized on a solid support, comprises the capture surface that could otherwise be utilized in a conventional sandwich ELISA method. In another example, in certain embodiments of the methods as otherwise described herein, the sample comprising an analyte bound to a capture reagent, the capture reagent immobilized on a microsphere, comprises the capture surface that could otherwise be utilized in a conventional suspension microarray method. In yet another example, in certain embodiments of the methods as otherwise described herein, the sample comprising an analyte immobilized on a cell surface or localized in a cell compartment comprises the cell sample that could otherwise be utilized in a conventional immunohistochemistry or immunofluorescence assay method. The person of ordinary skill in the art would appreciate that, in such embodiments, the analyte may be any antigen for which a conventional detection method exists, or for which a conventional detection method may be developed.

As described above, in various aspects of the methods of the disclosure, the method includes providing an enzyme having peroxidase-like activity (e.g., provided at a target site, the enzyme immobilized at a surface, or provided linked to a primary detection reagent, or in solution or suspension). In certain embodiments of the methods as otherwise described herein, the enzyme having peroxidase-like activity is a polypeptide. For example, in certain embodiments of the methods as otherwise described herein, the enzyme having peroxidase-like activity is a peroxidase, such as horseradish peroxidase (HRP). In other embodiments of the methods as otherwise described herein, the enzyme having peroxidase-like activity is a phosphatase, such as an alkaline phosphatase. In certain embodiments of the methods as otherwise described herein, the enzyme having peroxidase-like activity comprises a ribozyme or deoxyribozyme. The person of ordinary skill in the art will appreciate that other enzymes may provide sufficient peroxidase-like activity to catalyze the oxidative polymerization of polyphenols as described herein.

As described above, in various aspects of the methods of the disclosure, the method includes polymerizing (e.g., at the target site) a polyphenol (e.g., dopamine or a dopamine derivative). In certain embodiments of the methods as otherwise described herein, the polyphenol includes a fluorescent tag (e.g., a dopamine derivative including dopamine linked to a fluorescent tag). For example, in certain embodiments of the methods as otherwise described herein, the polyphenol includes a quantum dot (e.g., a dopamine derivative comprising dopamine linked to a quantum dot). In another example, in certain embodiments of the methods as otherwise described herein, the polyphenol includes a fluorescent dye (e.g., a dopamine derivative includes dopamine linked to a fluorescent dye). In certain embodiments of the methods as otherwise described herein, the polyphenol includes biotin (e.g., a dopamine derivative including dopamine linked to biotin). In certain embodiments of the methods as otherwise described herein, the method includes polymerizing, at the target site, the polyphenol (e.g., dopamine or a derivative thereof).

As described above, in various aspects of the methods of the disclosure, the method includes polymerizing (e.g., at a target site or otherwise), the polyphenol (e.g., dopamine or a dopamine derivative) in the presence of an oxidant. In certain embodiments of the methods as otherwise described herein, the oxidant is a peroxide such as hydrogen peroxide (H₂O₂). In other embodiments, other oxidants can be used, e.g., percarbonates.

As described above, in various aspects of the methods of the disclosure, the method includes polymerizing, at the target site, a polyphenol (e.g., dopamine or a dopamine derivative) in the presence of peroxide and an enzyme having peroxidase-like activity. In certain embodiments of the methods as otherwise described herein, the polymerization at the target site is further in the presence of a polypeptide (i.e., other than the enzyme having peroxidase-like activity). Without intending to be bound by theory, the present inventors believe that the polypeptide, comprising groups reactive with polyphenols and polyphenol polymers (e.g., dopamine, a dopamine derivative, and/or a polydopamine) serves to further enhance the polymerization and/or deposition rate of polyphenols in the presence of an oxidant and an enzyme having peroxidase-like activity. For example, in certain embodiments of the methods as otherwise described herein, the polymerization at the target site is further in the presence of bovine serum albumin (BSA). In certain embodiments of the methods as otherwise described herein, the polymerization at the target site is further in the presence of copper or iron. Without intending to be bound by theory, the present inventors believe that iron and/or copper serve to further enhance the polymerization rate of polyphenols derivative in the presence of an oxidant and an enzyme having peroxidase-like activity.

As described above, in various aspects of the methods of the disclosure, the method includes polymerizing (e.g., at a target site or otherwise) a polyphenol (e.g., dopamine or a dopamine derivative) in the presence of peroxide and an enzyme having peroxidase-like activity. In certain embodiments of the methods as otherwise described herein, the polymerization is in a buffer solution. For example, in certain embodiments of the methods as otherwise described herein, the polymerization at the target site is in a Tris buffer solution. In another example, in certain embodiments of the methods as otherwise described herein, the polymerization is in phosphate-buffered saline (PBS). In other embodiments, the buffer is a bicine buffer or a borate buffer. The person of ordinary skill in the art will appreciate that a variety of buffers can be used in the practice of the methods described herein. In certain embodiments of the methods as otherwise described herein, the polyphenol (e.g., dopamine or dopamine derivative) is present in the buffer solution in a concentration within the range of about 1 mM to about 200 mM. For example, in certain embodiments of the methods as otherwise described herein, the polyphenol (e.g., dopamine or dopamine derivative) is present in the buffer solution within the range of about 1 mM to about 190 mM, or about 1 mM to about 180 mM, or about 1 mM to about 170 mM, or about 1 mM to about 160 mM, or about 1 mM to about 150 mM, or about 1 mM to about 140 mM, or about 1 mM to about 130 mM, or about 1 mM to about 120 mM, or about 1 mM to about 110 mM, or about 1 mM to about 100 mM, or about 5 mM to about 200 mM, or about 10 mM to about 200 mM, or about 20 mM to about 200 mM, or about 30 mM to about 200 mM, or about 40 mM to about 200 mM, or about 50 mM to about 200 mM, or about 60 mM to about 200 mM, or about 70 mM to about 200 mM, or about 80 mM to about 200 mM, or about 90 mM to about 200 mM, or about 100 mM to about 200 mM.

As described above, in various aspects of the methods of the disclosure, the method includes polymerizing, at the target site, dopamine or a dopamine derivative in the presence of peroxide and an enzyme having peroxidase-like activity. In certain embodiments of the methods as otherwise described herein, the polyphenol polymer (e.g., polydopamine), deposited by the polymerization, has an optical density of at least about 0.05 at a wavelength of 450 nm or 700 nm. For example, in certain embodiments of the methods as otherwise described herein, the polyphenol polymer (e.g., polydopamine), deposited by the polymerization, has an optical density of at least about 0.1, or at least about 0.25, or at least about 0.5, at a wavelength of 450 or 700 nm (e.g., in a sample having a conventional path length). In certain embodiments of the methods as otherwise described herein, the polyphenol polymer (e.g., polydopamine), deposited by the polymerization, comprises an emission intensity of at least about 10 at a wavelength of 480 nm (e.g., at a conventional excitation wavelength).

Another aspect of the disclosure is an assay kit. In various aspects and embodiments, the disclosure demonstrates the kit to be compatible with virtually all common biodetection and bioimaging techniques. In certain embodiments of the kits as otherwise described herein, the kit includes a primary detection reagent linked to an enzyme having peroxidase-like activity, the primary detection reagent capable of binding an analyte, and dopamine or a dopamine derivative. In certain embodiments of the kits as otherwise described herein, the kit includes an intermediate detection reagent, capable of binding an analyte, a primary detection reagent linked to an enzyme having peroxidase-like activity, the primary detection reagent being capable of binding the intermediate detection reagent, and a polyphenol (e.g., dopamine or a dopamine derivative).

In certain embodiments of the kits as otherwise described herein, the polyphenol is linked to a fluorescent tag or biotin (e.g., a dopamine derivative including dopamine linked to a fluorescent tag or biotin). For example, in certain embodiments of the kits as otherwise described herein, polyphenol is linked to a quantum dot (e.g., a dopamine derivative including dopamine linked to a quantum dot). In another example, in certain embodiments of the kits as otherwise described herein, the polyphenol is linked to a fluorescent dye (e.g., a dopamine derivative including dopamine linked to a fluorescent dye). In certain embodiments of the kits as otherwise described herein, the kit further comprises a secondary detection reagent comprising an amine-functionalized tag or an enzyme capable of catalyzing the conversion of a chromogenic substrate. For example, in certain embodiments of the kits as otherwise described herein, the secondary detection reagent comprises an amine-functionalized quantum dot or an amine-functionalized fluorescent dye, e.g., Cy3. In another example, in certain embodiments of the kits as otherwise described herein, the secondary detection reagent comprises a polypeptide, e.g., horseradish peroxidase.

### EXAMPLES

The Examples that follow are illustrative of specific embodiments of the invention, and various uses thereof. They are set forth for explanatory purposes only, and are not to be taken as limiting the scope of the disclosure.

All chemicals and biochemicals (unless specified) were purchased from Sigma-Aldrich (St. Louis, Mo.) and used without further purification. 96-well plastic microplates (each microplate consists of twelve removable strips of wells and a frame) were purchased from R&D Systems (Minneapolis, Minn.). Nitrocellulose membranes were purchased from EMD Millipore (Billerica, Mass.uman cervical cancer (HeLa) cell line was purchased from ATCC (Manassas, Va.). Glass-bottom 24-well plates (black wall) were purchased from Greiner Bio-One (Monroe, N.C.). Fetal bovine serum was purchased from FAA laboratories (Dartmouth, Mass.). Casein (5% solution) was purchased from Novagen (Billerica, Mass.). Anti-HSP90 antibody raised in rabbit (LOT: SAB4300541), anti-Lamin A antibody raised in rabbit (LOT: L1293), and anti-GAPDH antibody raised in rabbit (LOT: G9545) were purchased from Sigma-Aldrich (St. Louis, Mo.). CRHR1/CRF1 antibody was purchased from Novas Biologicals (LOT: NLS1778, Littleton, Colo.). Monoclonal rabbit antibodies raised against Ki-67 was purchased from Epitomics (LOT: 42031, Burlingame, Calif.). Monoclonal rabbit antibodies against Cox4 (REF: 4850s), and mouse programmed death ligand-1 expression (PD-L1) (REF: 29122S) were purchased from Cell signaling Technology (Danvers, Mass.). Goat anti-rabbit IgG (H+L) HRP-2′Ab (LOT: RA230590), goat anti-mouse IgG (H+L) HRP-2′Ab (LOT: 31430), nitrocellulose membranes for dot-blotting (0.45 11m pore size) with high binding affinity, MEM culture medium with L-glutamine, Pierce™ DAB Substrate Kit, QDs (525 nm emission) functionalized with secondary Ab fragments (Qdot goat F(ab′)2 anti-rabbit IgG conjugates (H+L)) (LOT: 1738599), amine-functionalized QDs (Qdot® 525 ITK™ Amino (PEG) Quantum Dots) (LOT: 1763984), amine functionalized QDs (Qdot® 605 ITK™ Amino (PEG) Quantum Dots) (LOT: 1630058), streptavidin functionalized QDs (Qdot® 605 Streptavidin Conjugate) (LOT: Q10101MP), and HRP-conjugated streptavidin (LOT: 1012719A) were purchased from ThermoFisher. Cy3 labelled donkey anti-mouse IgG (H+L) (LOT: 715165150) and Cy3 labelled donkey anti-rabbit IgG (H+L) (LOT: 711165152) were purchased from Jackson ImmunoResearch Laboratories (West Grove, Pa.). Fluorescent beads (carboxylic groups on surface) 5 11 m in diameter with three colors (green 480/520nm excitation/emission maxima, yellow 525/565nm, red 660/690 nm) were purchased from Bangs Laboratories (LOT: 11534; 9920; 11376, Fishers, IN). All antibodies were obtained in PBS without carrier proteins or stabilizing reagents. Mouse IgG, HIV p24, KLK3, CRP and VEGF ELISA kits were either purchased from Abeam (REF: ab151276, Cambridge, Mass.) or R&D Systems (LOT: DHP240; DKK300; DCRPOO; DVEOO). Seroconversion plasma samples from HIV infected patients were purchased from SeraCare (LOT: 06000237; 06000230; 06000227; 06000262, Milford, Mass.). Serial bleeds were collected from patients during the development of an HIV infection. All HIV patients' plasma samples were tested and found negative to HBsAg and HCV. Heathy patient plasma samples (age, 25-65) were purchased from Discovery Life Sciences (Los Osos, Calf.). All plasma samples were tested and found negative to HBV, HCV, HIV and RPR.

### Example 1

### Enzyme-Accelerated Ultrafast PDA Deposition

To quantify the effect of HRP on FDA polymerization rate, the enzyme-accelerated signal enhancement (EASE) process is compared to the reaction conditions in the conventional dip-coating polymerization procedure where HRP is not present and O₂ is the oxidant.

Preparation of dopamine solution for EASE. Dopamine hydrochloride powder (15 mg) was dissolved rapidly in tris buffer (10 mM, 3 ml) at pH 8.5, followed by quick addition of H₂O₂ (1M, 60 μl). The mixture solution was used fresh.

Polydopamine deposition. Small droplets of HRP (0.1 pg) in PBS buffer and/or BSA (15 pg) in PBS buffer were placed on a nitrocellulose membrane and air-dried for 1 hour at room temperature. The membranes were further exposed to the EASE assay for 1 minute and washed with PBS for 30 seconds.

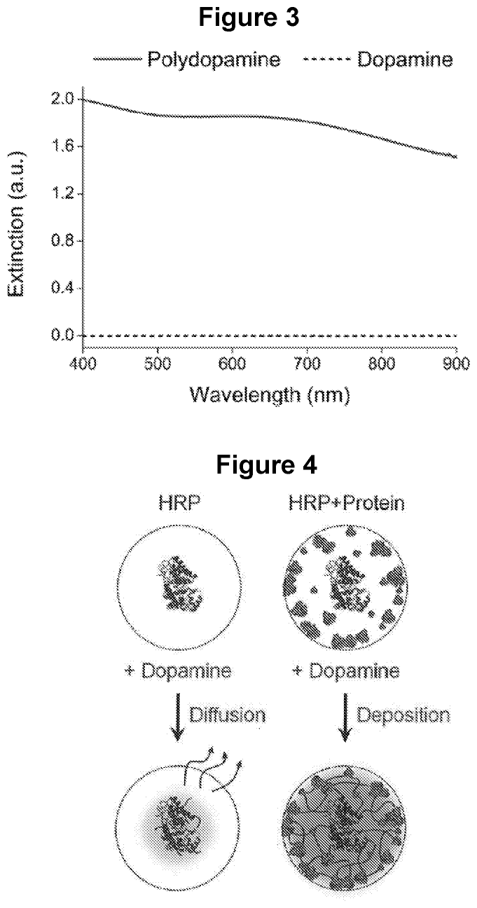

Results. As shown in FIG. 2A, the dopamine solution slowly changed color from colorless to light grey over a period of four hours, indicating slow FDA formation. In contrast, when HRP and H₂O₂ of low concentration (typical reaction condition for HRP-catalyzed substrate conversion) were added, the dopamine solution of the same concentration instantly turned to brown-black, showing significantly increased PDA polymerization rate. Quantitative comparison of the reaction kinetics was plotted by measuring the solution light extinction at 700 nm where dopamine has negligible absorption compared to PDA (FIG. 3). Under the conventional dip-coating reaction conditions, PDA slowly built up and was not near completion after 4 h of reaction time; whereas under the EASE condition, the FDA solution reached the same level of light extinction in 48 seconds (plateaued within 1 h), indicating an approximately 300-fold increase in polymerization rate (FIG. 2B).

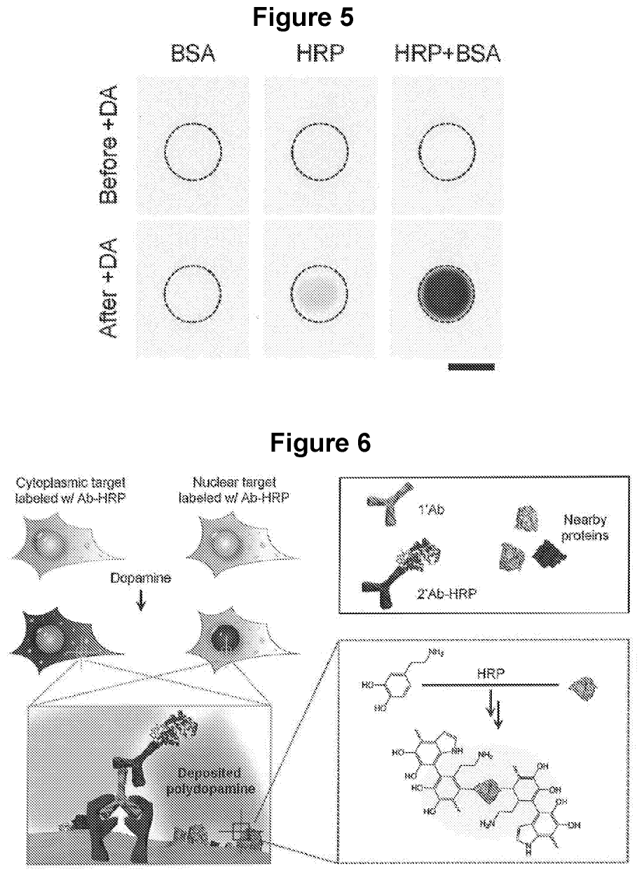

Next, it was determined whether the EASE process can be confined to the vicinity of HRP molecules (FIG. 4), a key factor determining the scope of downstream applications. If FDA molecules quickly diffuse away from HRP, the EASE technology would only be useful for improving the enzyme-linked immunosorbent assay (ELISA) by measuring chromogens in solution. If the FDA molecules are confined near HRP, the EASE technology will be broadly applicable to various bioassays beyond ELISA, such as immunohistochemistry (IHC), immunofluorescence (IF), fluorescence in situ hybridization (FISH), and immunoblotting, because the spatial information is preserved. To determine this, HRP was immobilized inside a circle on a nitrocellulose membrane, which was also blocked with a polypeptide, bovine serum albumin (BSA). Note that BSA, as a standard blocking agent that helps reduce non-specific binding, can serve an additional function. It can provide reactive chemical groups that function as FDA deposition anchor sites. As shown in FIG. 5, when the membrane was exposed to dopamine/H₂O₂ solution, essentially no PDA was found on the membrane with BSA only (free of background). In contrast, when HRP is present on the membrane, PDA development became clearly visible, because HRP not only catalyzes the FDA polymerization, but also, as a polypeptide, could serve as a FDA deposition anchor point. For the membrane incubated with HRP and blocked with BSA, PDA deposition was significantly enhanced due to the high density of reactive sites on the membrane (provided by the BSA molecules) that quickly captured PDA molecules before they diffused away from the surface. More importantly, the color development was completely confined inside the HRP spot, demonstrating retention of the spatial resolution that makes EASE suited for the aforementioned immuno and hybridization assays.

### Example 2

### EASE for Immunohistochemistry and Immunofluorescence

The EASE technology was first applied to IHC and IF, robust technologies capable of interrogating gene expressions in single cells and resolving the heterogeneity issues of complex tissue samples, with well-preserved cell and tissue morphology. IHC and IF work well for high-abundance analyte molecules, but lack the sensitivity to detect antigens of low abundance, in particular in clinical tissue specimens where autofluorescence can be overwhelmingly high. To test the suitability of EASE, two model antigens, Lamin A (nuclear envelope) and HSP-90 (cytoplasm) were stained in formalin-fixed HeLa cells because these two antigens represent analytes in different cell compartments (FIG. 6). Conventional two-step staining procedure was carried out by incubating cells with an intermediate detection reagent (primary antibody (1′Ab)) and a primary detection reagent (secondary antibody-HRP (2′Ab-HRP)), sequentially, except that dopamine was used as the HRP substrate. Owing to the chromogenic feature of PDA, the staining can be directly visualized.

Cell culture and fixation. HeLa cells were cultured in MEM medium with L-glutamine, 10% fetal bovine serum, and antibiotics (60 μg ml-1 streptomycin and 60 U ml-1 penicillin) in glass-bottom 24-well plates to 60-80% confluency. Before IF staining, cells were rinsed with 1× tris-buffered saline (TBS), fixed with 4% formaldehyde in TBS for 30 minutes, permeabilized with 2% DTAC (dodecyltrimethylammonium chloride)/TBS for 30 minutes followed by 0.25% TritonX-100/TBS for 5 minutes and washed five times with TBS (each time 3 minutes). The fixed cells were stored in 1×PBS at 4′C.

Cell imaging and signal analysis. An Olympus IX-71 inverted fluorescence microscope equipped with a true-color charge-coupled device (QColor5, Olympus), a LSM 510 Meta confocal microscope (Zeiss, Dublin, Calif.) and a hyper-spectral imaging camera (Nuance, 420-720 nm spectral range, CRI, now Advanced Molecular Vision) were used for cell imaging. Low-magnification images were obtained with a 20× objective (NA 0.75, Olympus) and high-magnification with 40× and 100× oil-immersion objectives (NA 1.40, Olympus). Wide UV filter cube (330-385 nm band-pass excitation, 420 nm long-pass emission, Olympus) was used for imaging of all QD probes. All images were acquired with cells attached to the coverslip bottom of the well and immersed in PBS without anti-fading reagents. For quantitative comparisons, the same exposure time and gain were applied during imaging. Nuance image analysis software and lmageJ were used to identify regions of interest that included stained cells and excluded ‘blank’ cell-free areas. Average fluorescence intensity throughout all regions of interest within a single image was recorded. Identical analysis was performed on 4 images (containing ˜40 cells per field of view) taken from different areas of the sample to obtain an overall average staining intensity and assess signal variation.

IHC/IF-EASE single cell imaging. Prior to staining, the endogenous peroxidase activity of cells was quenched by 3% H₂O₂ solution. Cells were first blocked with 2% BSA/0.1% casein in 1× PBS for 30 minutes. Rabbit anti-Lamin A IgG (LOT: L1293, Sigma-Aldrich) or anti-HSP90 IgG (LOT: SAB4300541, Sigma-Aldrich) (intermediate detection reagent) diluted in PBS buffer containing 6% BSA was added to the cells. After 1-hour incubation, cells were washed three times (5 minutes each) with PBS containing 2% BSA, followed by another 1-hour incubation with goat anti-rabbit IgG (H+L) HRP-2′Ab (LOT: RA230590, ThermoFisher) (primary detection reagent). Unbound antibodies were washed away with PBS with 2% BSA (5 min×3), and fresh enzyme substrate (dopamine or DAB) was added to cells for 15 minutes incubation. The ideal staining result is strong chromogen signal of interested analyte locations with low nonspecific signals in background. To characterize the staining stability after storage, the stained cells were stored in 1×PBS at 4° C., and washed with fresh PBS every four days. Images were captured every three weeks on the same cell subset with the same exposure and gain. For immunofluorescence imaging with a secondary detection reagent (QDs), after the PDA development step, amine-functionalized PEG-coated QDs (10 nM) were incubated with cells for 1 hour.

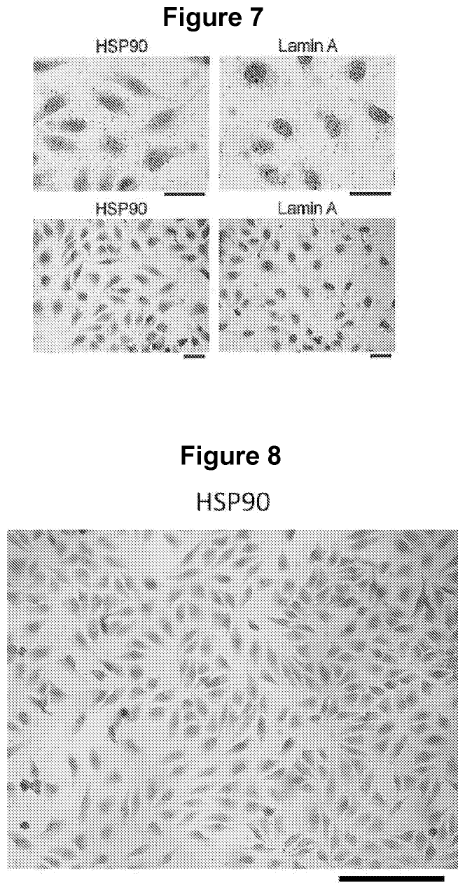

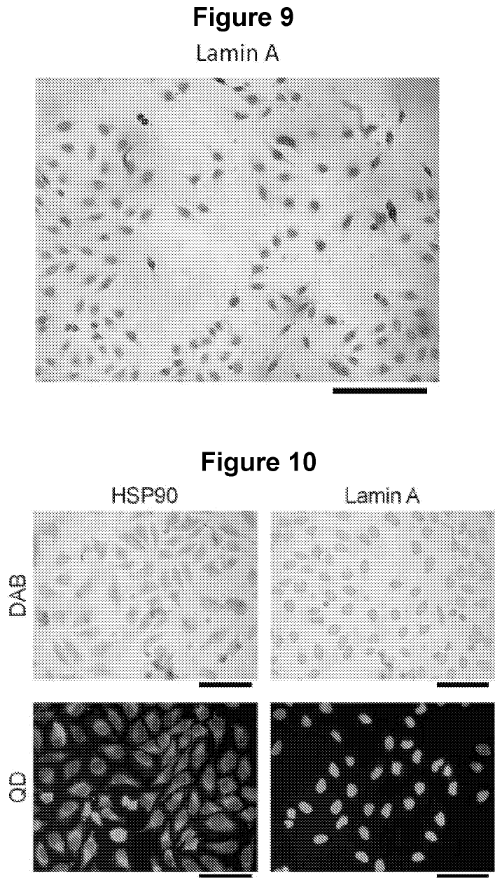

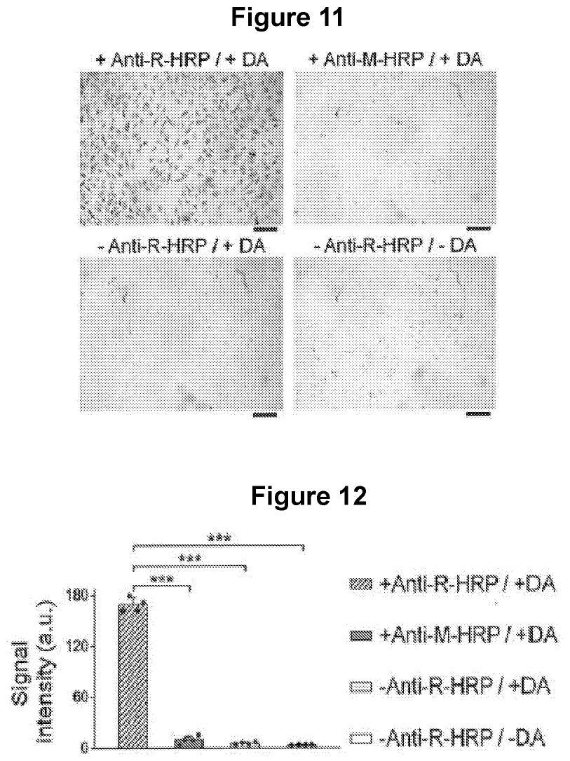

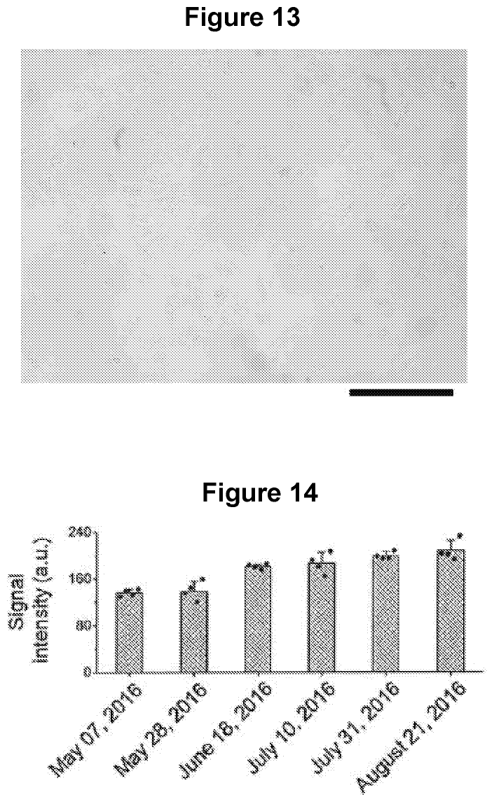

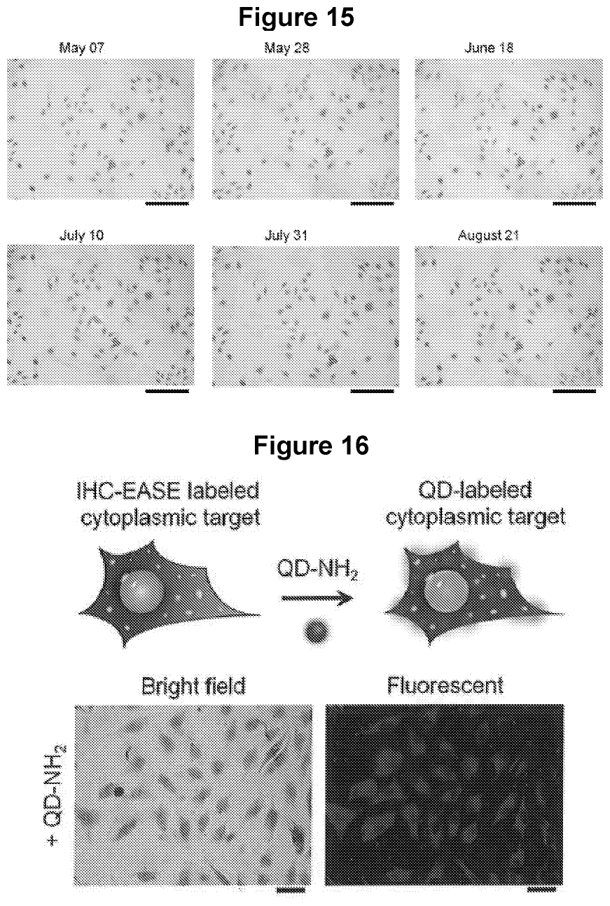

Results. As shown in FIGS. 7-9, the staining patterns for both antigens were the same as those obtained with conventional IHC (using 3,3′-diaminobenzidine (DAB) as the substrate) and IF (using quantum dot (QD) labeled secondary antibody) (FIG. 10), demonstrating the staining specificity and confirming confined PDA deposition on the microscopic scale. The specificity was further confirmed by a series of control experiments where either one of the key agents (1′Ab and 2′Ab-HRP) was missing or a mismatched 1′Ab-2′Ab pair was used (FIGS. 11-13). The PDA chromogens were highly stable after cell staining. As shown in the same group of cells in FIGS. 14-15, no obvious signal decay was detected after 4 months, allowing samples to be reexamined after extended storage. In fact, the signal slightly increased—without intending to be bound by theory, the present inventors believe the increase to be due to aging of the rapidly formed PDA.

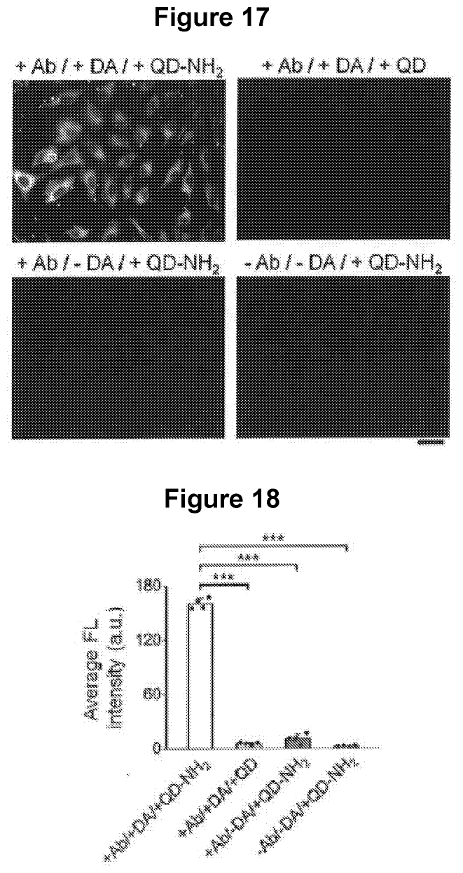

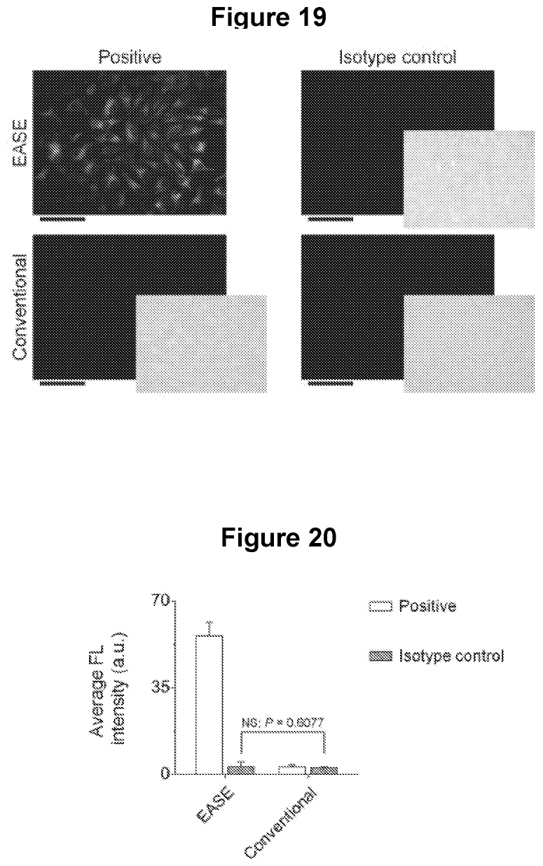

To probe the sensitivity enhancement of EASE, fluorescence probes (secondary detection reagents) were brought into the assay after PDA deposition, taking advantage of PDA's remarkable reactivity to any fluorophores with primary amines and the convenience of quantifying fluorescence signals. Pegylated QDs with terminal amines were used as the fluorophore because of their photostability, which allows for accurate measurement of fluorescence intensity. As shown in FIG. 16, the fluorescent staining pattern matched that of the PDA, confirming that QD-N H2 immobilization was confined to the PDA network. The specificity was further demonstrated by the control experiments where either one of the key agents (Ab or dopamine) was missing, an isotype 1′Ab was utilized, or non-functionalized QDs were used. As shown in FIGS. 17-20, the control experiments did not produce detectable signals.

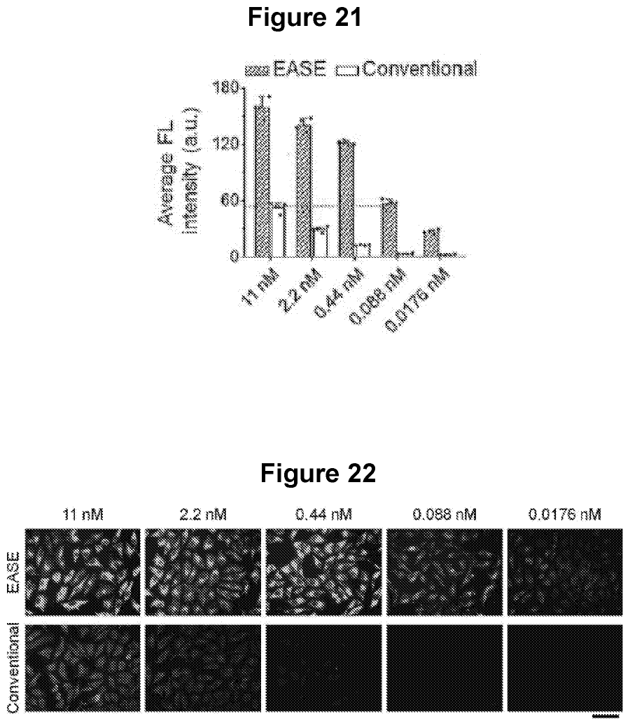

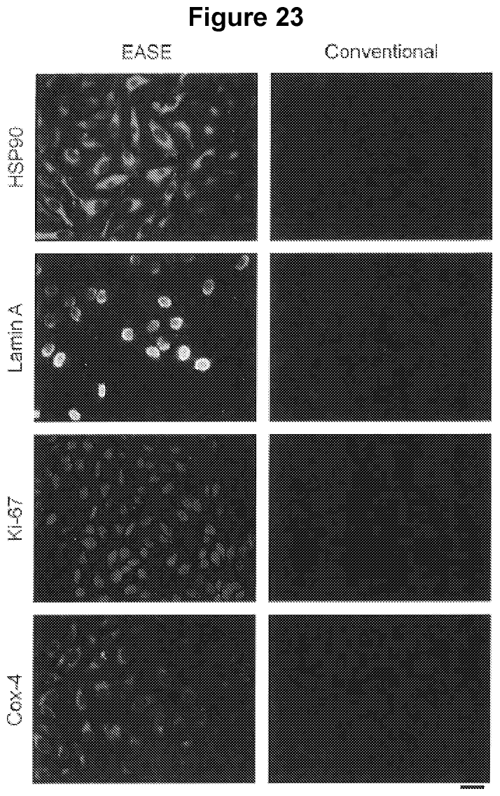

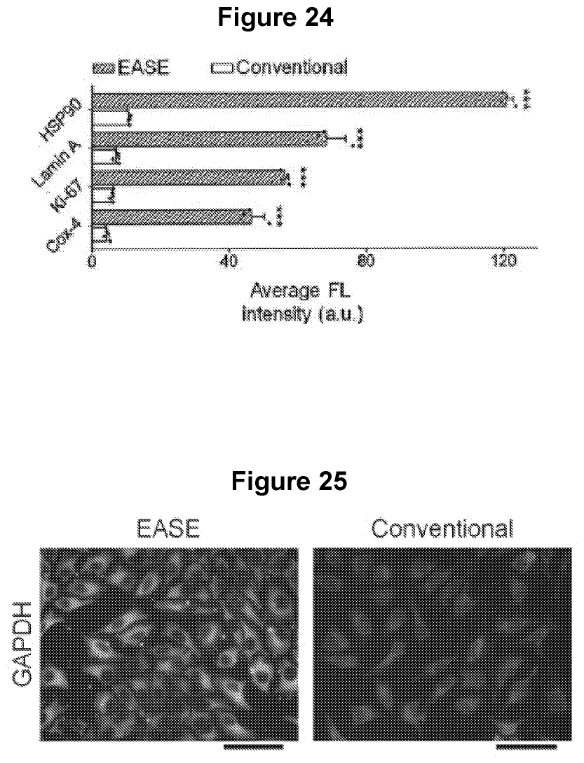

To evaluate the sensitivity quantitatively, staining was first performed on HSP-90. Unlike ELISA assays where analyte molecules can be easily immobilized at various densities, engineering cells with a variety of precisely controlled antigen expression levels is extremely difficult. Instead, the concentration of the intermediate detection reagent (1′Ab) was reduced in a serial fashion to bring down the signal intensity. As shown in FIGS. 21-22, at a 1′Ab concentration of 88 pM, IF-EASE achieves the same signal strength compared to conventional IF at 1′Ab of 11 nM, yielding a 125 fold reduction in 1′Ab concentration, which not only demonstrates enhancement of imaging sensitivity, but also demonstrated the ability of EASE to reduce the cost of expensive biological agents such as antibodies. The signal enhancement was a result of amplifying a limited number of analyte molecules (as well as bound HRP) to a polymer network that captures a large number of QDs. Indeed, IF-EASE of four tumor biomarkers (HSP90, Lamin A, Ki-67, and Cox-4) covering various intracellular locations, at a 1:25,000 dilution of the primary antibodies (typical IF dilution factor ˜1:100), produced bright and specific staining similar to those from conventional IF assay using high concentration of 1′Ab (FIGS. 23-24). In contrast, without EASE, 1:25,000 dilutions of the primary antibodies did not produce detectable signals.

Next, to directly evaluate IF-EASE in imaging low-abundance analytes, the expression of GAPDH in HeLa cells was silenced using RNA interference (RNAi)-mediated gene knockdown. RNA interference. GAPDH expression knock-down was done by transfecting siRNA targeting GAPDH into HeLa cells. Annealed siRNA with 3′-TT overhangs was purchased from IDT (Coralville, Iowa). The sense strand sequence was 5′-CAUCAUCCCUGCCUCUACUTT-3′. HeLa cells were grown in a 10 cm TC-treated dish, trypsinized, and mixed in suspension with culture medium containing 25 nM GAPDH siRNA, together with 0.5 μl per well DharmaFECT-2 transfection reagent (Dharmacon). The cells (500 μl cell suspension per well) were then seeded into a glass-bottom 24-well plate, and incubated for 36 or 60 hours. Following RNAi, the cells were processed for staining using IF-EASE. The intermediate detection reagent (1′Ab) was anti-GAPDH (rabbit, LOT: G9545, Sigma-Aldrich).

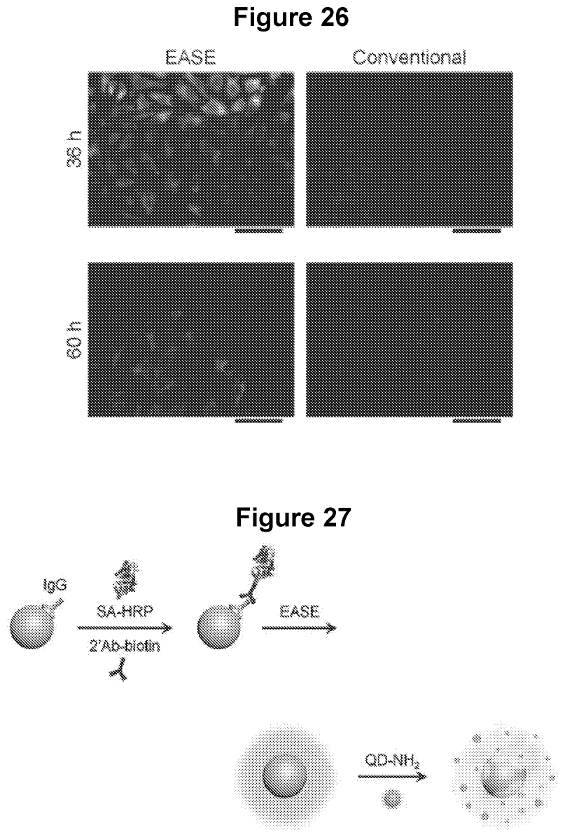

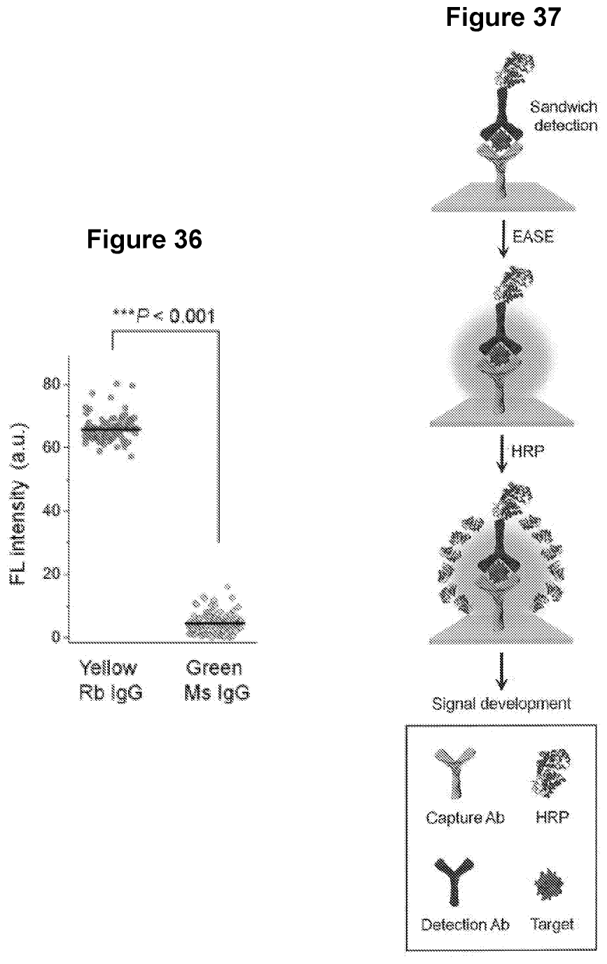

Results. As shown in FIGS. 25-26, 36 h post RNAi, the characteristic cytoplasmic distribution of GAPDH could be clearly visualized using IF-EASE, but was only barely detectable using IF alone. Similarly, at 60 h post RNAi, trace amount of GAPDH could still be detected using IF-EASE, but not with IF alone. This result clearly demonstrates the power of EASE in detection of low-abundance analytes in cells.

### Example 3

### EASE for Suspension Microarrays

Suspension microarrays are highly multiplexed genotyping and phenotyping platforms used in molecular biology, drug screening, and disease diagnosis. Compared to planar microarrays that are spatially addressable, suspension microarrays are often fabricated by doping microspheres with combinations of luminescent materials and are decoded with flow cytometers (e.g., Luminex microbeads). To determine whether an unknown analyte is present or not, conventional methodologies such as direct or sandwich hybridization and immuno-recognition are applied. The suspension microarrays offer advantages such as faster binding kinetics, but their detection sensitivities are essentially the same as the planar counterparts.

Preparation of antigen-coated fluorescent beads. IgG purified from mouse and rabbit serum (capture reagent) were covalently linked to the surface of green and yellow fluorescent beads, via 2-step carbodiimide-mediated cross-linking between the carboxylic groups on bead surface and the primary amines on IgG. Briefly, fluorescent beads were first washed and suspended in MES buffer (pH 4.8) with 0.01% Tween-20 at 0.1 w/v% (˜107 beads ml-1) and activated for 15 minutes upon addition of 10 mg ml-1 1-Ethyl-3-(3-dimethylaminopropyl)carbodiimide (EDC) and 10 mg ml-1 N-hydroxysulfosuccinimide (sulfa-NHS). The activated beads were washed by centrifugation (5,000 g×2 min) twice using 50 mM borate buffer (pH 8.5) with 0.01% Tween-20 to remove excess crosslinkers and then incubated with IgG (2.5 mg ml-1) in borate buffer with 0.01% Tween-20 for 6 hours. The resulting IgG-coated beads were washed 3 times to remove excess IgG, resuspended in PBS (with 0.5% BSA), and stored at 4° C.

Suspension microarray with EASE. Biotinylated goat anti-mouse and goat anti-rabbit IgGs (model analytes) were captured by the antibody-coated green and yellow beads. PBS containing 0.5% BSA was used as incubation and blocking buffer throughout the experiment. All incubation steps were carried out at room temperature under gentle rotation. All washing steps were done by centrifuging the microbeads at 3,000 g for 2 minutes. Each microbead type was resuspended in 100 μl PBS at a final concentration of 1×106 beads ml⁻¹. The beads were first incubated in the blocking buffer for 30 minutes. Biotinylated anti-mouse or -rabbit IgGs were added to the bead solution, incubated for 30 minutes, washed 3 times with PBS (0.5% BSA), and resuspended in 100 μl buffer. Then HRP-streptavidin probes (primary detection reagent) (1:3000 dilution) were added to the bead solution, incubated for 30 minutes, washed 3 times with PBS (0.5% BSA), resuspended in 100 μl dopamine solution for EASE, followed by 15 min incubation. The microbeads were washed another 3 times in BSA-free PBS, and mixed with amine-functionalized PEG-coated QDs (secondary detection reagent) (1 nM final concentration) for 1 hour incubation. At the end of QD incubation, the beads were washed 5 times with DI water and concentrated in 10 μl water for microscopy examination. A hyper-spectral imaging camera (Nuance, 420-720 nm spectral range, CRI, now Advanced Molecular Vision) and software were used to unmix and quantify fluorescence signal components. False-color composite images were obtained by merging individual channels. For quantitative analysis, Nuance image analysis software was used to automatically identify regions of interest that included QD labelling. Identical analysis was performed on 5 images (containing at least 20 beads per field of view). High-throughput quantitative analysis was achieved on a LSR-II flow cytometer (BD Biosciences). For each sample, at least 5,000 beads were counted. The flow cytometry data was analyzed with FlowJo 9.3.3 (TreeStar).

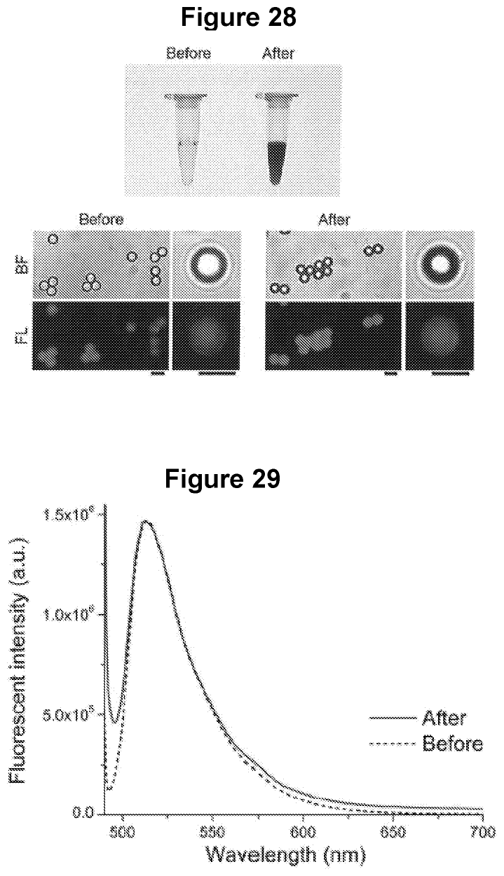

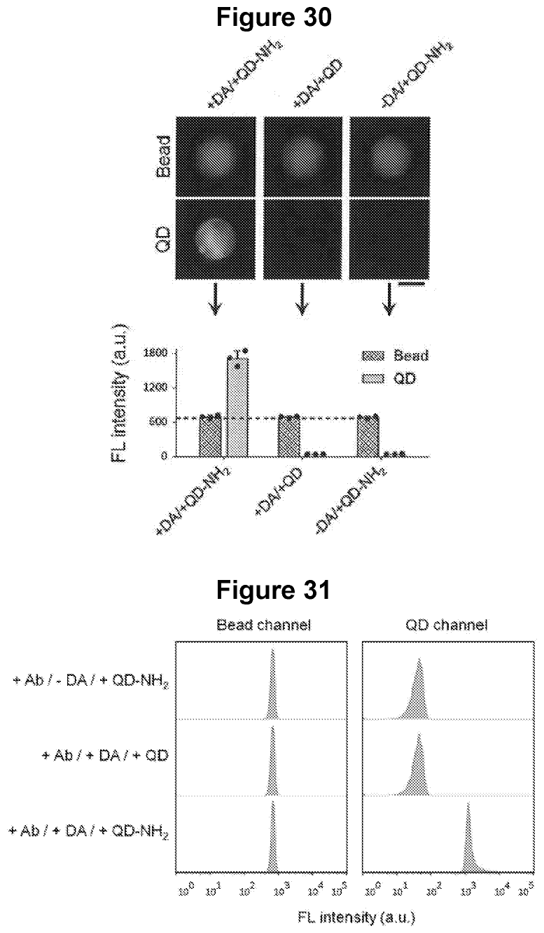

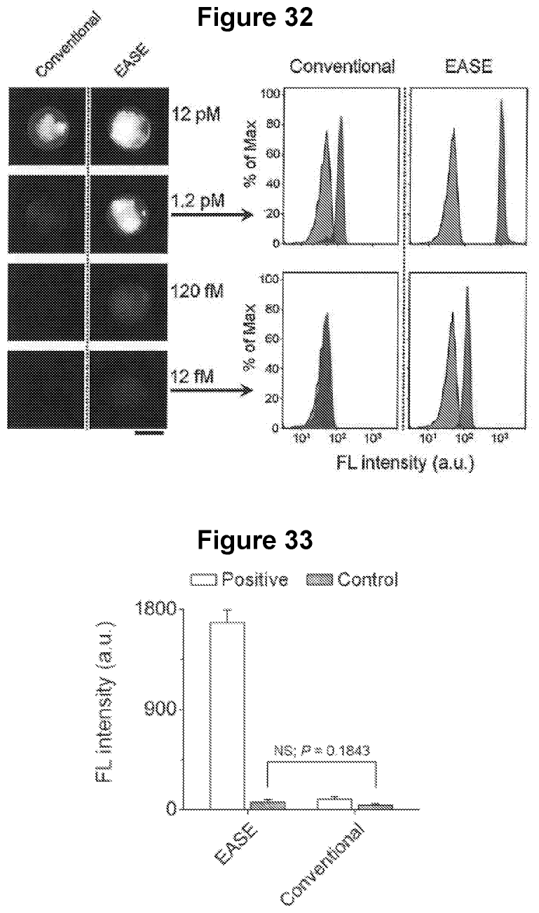

Results. To demonstrate the compatibility of EASE with suspension microarrays, fluorescent microspheres were coated with immunoglobulin G (IgG) (capture reagent) to detect a model analyte, biotinylated 2′Ab. Presence or absence of the analyte was detected with either streptavidin-QD conjugates (conventional sandwich method) or the EASE technology (primary detection reagent (streptavidin-HRP), PDA, and secondary detection reagent (QD-NH₂)) (FIG. 27). Before comparing their sensitivities, it was determined whether PDA deposition on microsphere surface reduced the microsphere fluorescence (which would interfere with fluorescence barcodes if multiple colors were doped inside). PDA coating on microsphere was easy to monitor because the solution quickly turned dark brown due to chromogenic PDA (FIG. 28), yet microscopy images revealed virtually no change of the microsphere fluorescence before and after PDA coating. Additional quantitative evaluation of the microspheres using a fluorometer unambiguously confirmed the microscopy result (FIG. 29). QDs were used as the fluorescent secondary detection reagent because of their tunable fluorescence emission and the large Stokes shift (to avoid spectral overlap with the microsphere fluorescence). For QDs with various surface chemistries, only aminated QDs bound to the microspheres, showing that the interaction is due to the chemical reactions between amines and FDA, rather than physical adsorption (FIGS. 30-31). Next, the sensitivity was measured. Flow cytometry and fluorescence microscopy both revealed that the analyte IgG could be detected at a concentration of 1.2 pM using conventional sandwich assay (streptavidin-QD as the reporter), whereas addition of EASE could lower the detection limit by 2 orders of magnitude (fM range) (FIG. 32).

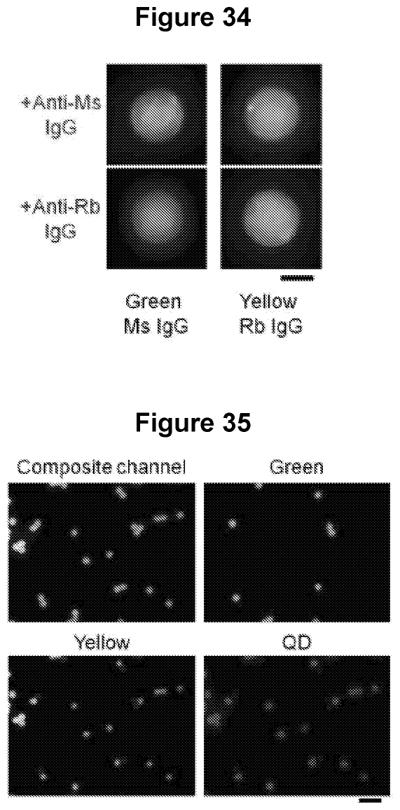

To assess the specificity of this ultrasensitive detection assay, two control experiments were conducted. In the first experiment where the analyte molecule was missing, no significant signals were detected with or without the EASE process, confirming the antibody-antigen binding specificity (FIG. 33). Second, potential crosstalk was evaluated using a dual-color setup. Two types of microspheres were mixed together, green microsphere with mouse IgG on the surface and yellow microsphere with rabbit IgG. When anti-rabbit IgG was added as the analyte, strong fluorescence signal from the EASE assay was only detected on the yellow microspheres, free of crosstalk (FIGS. 34-36). This remarkable detection specificity lays the foundation for massive parallel screening applications with additional optical barcodes.

### Example 4

### EASE for ELISA and Lateral Flow Strips

To demonstrate the versatility of EASE, it was further applied to ELISA and immuno strip tests, robust and poplar biochemical assays. These assays using antibodies for molecular recognition and enzyme-catalyzed chromogen development for analyte identification are easy to perform, having broad applications in both research and clinical laboratories. On the other hand, their mediocre detection sensitivities are also well acknowledged. Compared to the suspension assays discussed above, a technical feature of these assays is that they are performed on solid supports (flat surfaces or porous membranes), rendering the sample washing steps quick and easy (dip in and out of washing buffer without the need of a centrifuge). This seemingly insignificant feature, combined with the unique bioconjugation capability of FDA allows EASE to be carried over for more than one time. For example, in the first round of amplification, HRP molecules bound to the analyte can catalyze localized deposition of FDA. The FDA layer can in turn capture a large number of HRP molecules that are capable of catalyzing the conversion of chromogenic substrates (FIG. 37). ELISA-EASE. Mouse IgG, HIV p24, KLK3, CRP and VEGF (commercial kits purchased from Abcam (REF: ab151276, Cambridge, Mass.) or R&D Systems (LOT: DHP240; DKK300; DCRP00; DVE00)) were used as model analytes for the ELISA experiments. 96-well plastic plates coated with capture antibodies (capture reagents) were first blocked with PBS containing 2% BSA. 200 μl samples with serial dilutions and control samples were added into different wells. The wells were covered with adhesive strips and incubated for 2 hours at room temperature, washed 4 times, incubated with Ab-HRP conjugates (primary detection reagents) for 2 hours at room temperature, washed 4 times with PBS (6% BSA), incubated with dopamine solution for 15 minutes, washed 3 times with PBS, incubated with HRP (1 nM) in PBS for 1 hour, and washed 4 times with PBS (6% BSA). 200 μl of the substrate solution was added to each well and the reaction was quenched after 20 min incubation in dark. Absorbance at 450 nm (optical density) was measured using an Infinite M 200 plate reader (Tecan). The results were compared with those obtained with conventional ELISA assays.

The sensitivity of ELISA-EASE in detecting HIV p24 in plasma was probed by spiking HIV p24 of known concentrations into plasma from healthy donors. For plasma samples from both HIV infected patients and healthy donors, immune complex disruption and neutralization procedures were applied to treat the samples. 20 μl 5% Triton X-100, 90 μl plasma samples, 90 μl glycine reagent (1.5 M) were mixed and incubated for 1 hour at 37° C. 90 pl tris buffer (1.5 M) was then added into the mixed solution and incubated for 10 minutes at room temperature. The plasma samples from HIV-positive groups with high HIV p24 concentration were diluted (10× and 100×) to fit within the ELISA working ranges for measurement.

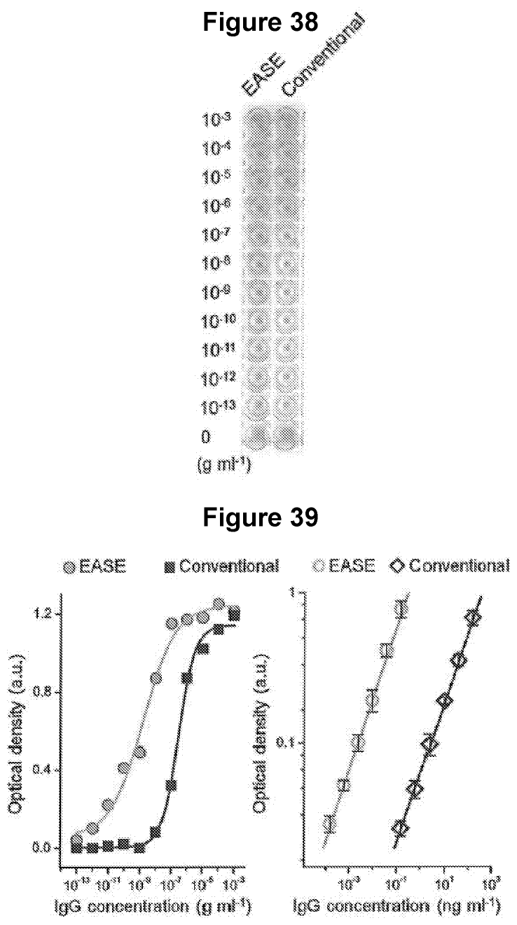

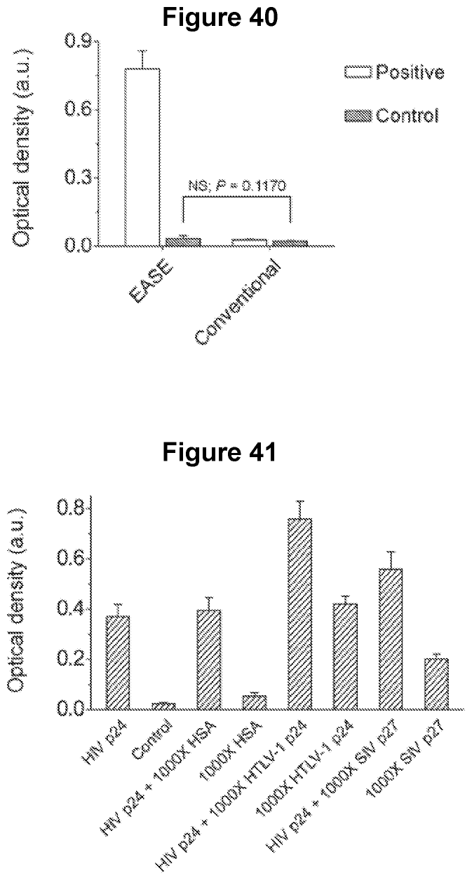

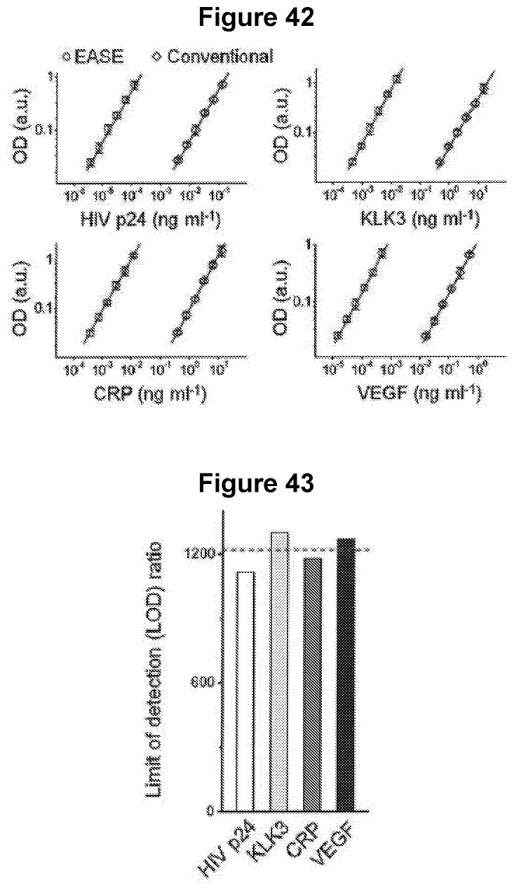

Results. To probe the sensitivity and specificity of ELISA with or without EASE, a standard sandwich ELISA assay was established to detect mouse IgG (model analyte). Serial dilution of the analyte molecule resulted in gradients of color development that could be easily visualized by naked eye (substrate: tetramethylbenzidine or TMB). As shown in FIG. 38, without EASE, color development in the ELISA assay was visible at an analyte concentration between 10⁻⁷ and 10⁻⁸ g m1⁻¹; with EASE as an add-on step, the color development became clearly visible at 10⁻¹² g ml⁻¹. This significantly improved limit of detection (LOD) was further quantified on a plate reader. The standard curve relating signal strength and analyte concentration is shown in FIG. 39 (left panel), with a zoomed-in low-concentration range plotted in the right panel. The plate-reader readouts reveal that the ELISA LODs (3 s.d. from the background) were 85.3 fg ml⁻¹ (with EASE) and 108 pg ml⁻¹ (without EASE), a 1,266-fold improvement. The specificity of the ELISA assays was demonstrated by control experiments where the analyte molecule was missing (FIG. 40) or high-concentration non-target analytes were introduced (FIG. 41). The robustness of the EASE-aided ELISA was further demonstrated with another four disease biomarkers: HIV capsid antigen p24 (HIV p24), kallikrein 3 (KLK3), c-reactive protein (CRP), and vascular endothelial growth factor (VEGF). Similarly, their calculated values of LODs of ELISA-EASE were 2.87 fg ml⁻¹, 0.31 pg ml⁻¹, 0.24 pg ml⁻¹, and 11.5 fg ml⁻¹ (FIG. 41 and Tables 1-4), respectively, representing an average 1,217-fold improvement over the conventional ELISA (FIG. 43).

TABLE 1
HIV p24 ELISA Data

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.81E−06 | 0.0251 | 0.0042 | 0.0039 | 0.027 | 0.003 |
| 7.63E−06 | 0.0467 | 0.0099 | 0.0078 | 0.0524 | 0.005 |
| 1.53E−05 | 0.1052 | 0.0205 | 0.0156 | 0.0995 | 0.0205 |
| 3.05E−05 | 0.188 | 0.0237 | 0.0313 | 0.2081 | 0.0196 |
| 6.10E−05 | 0.3706 | 0.0469 | 0.0625 | 0.3698 | 0.0298 |
| 1.22E−04 | 0.6985 | 0.1253 | 0.125 | 0.7083 | 0.0693 |

TABLE 2
KLK3 ELISA Data

| EASE | | | | | |
|---|---|---|---|---|---|
| KLK3 | | | Conventional | | |
| (ng ml⁻¹) | OD | S.D. | (ng ml⁻¹) | OD | S.D. |
| 4.59E−04 | 0.0262 | 0.0033 | 0.4688 | 0.0246 | 0.0023 |
| 9.18E−04 | 0.0531 | 0.0049 | 0.9375 | 0.0509 | 0.0045 |
| 0.0018 | 0.1196 | 0.0291 | 1.875 | 0.0987 | 0.0123 |
| 0.0037 | 0.273 | 0.043 | 3.75 | 0.1966 | 0.0184 |
| 0.0073 | 0.5882 | 0.0581 | 7.5 | 0.379 | 0.0349 |
| 0.0147 | 1.1994 | 0.1967 | 15 | 0.7601 | 0.16 |

TABLE 3
CRP ELISA Data

| EASE | | | | | |
|---|---|---|---|---|---|
| CRP | | | Conventional | | |
| (ng ml⁻¹) | OD | S.D. | (ng ml⁻¹) | OD | S.D. |
| 3.81E−04 | 0.0312 | 0.0039 | 0.3905 | 0.0323 | 0.0026 |
| 7.63E−04 | 0.0662 | 0.0105 | 0.781 | 0.072 | 0.0098 |
| 1.53E−03 | 0.1311 | 0.016 | 1.562 | 0.1531 | 0.0155 |
| 3.05E−03 | 0.2998 | 0.062 | 3.124 | 0.3697 | 0.0377 |
| 6.10E−03 | 0.5913 | 0.1238 | 6.248 | 0.7716 | 0.0992 |
| 1.22E−02 | 1.2022 | 0.0929 | 12.496 | 1.5002 | 0.3503 |

TABLE 4
VEGF ELISA Data

| EASE | | | | | |
|---|---|---|---|---|---|
| VEGF | | | Conventional | | |
| (ng ml⁻¹) | OD | S.D. | (ng ml⁻¹) | OD | S.D. |
| 1.53E−05 | 0.026 | 0.003 | 0.0157 | 0.0253 | 0.0021 |
| 3.06E−05 | 0.0504 | 0.0053 | 0.0313 | 0.046 | 0.0055 |
| 6.11E−05 | 0.0925 | 0.019 | 0.0625 | 0.0902 | 0.0067 |
| 1.22E−04 | 0.1789 | 0.02 | 0.125 | 0.169 | 0.0122 |
| 2.45E−04 | 0.3297 | 0.0401 | 0.25 | 0.3266 | 0.0702 |
| 4.89E−04 | 0.719 | 0.1208 | 0.5 | 0.6594 | 0.0528 |

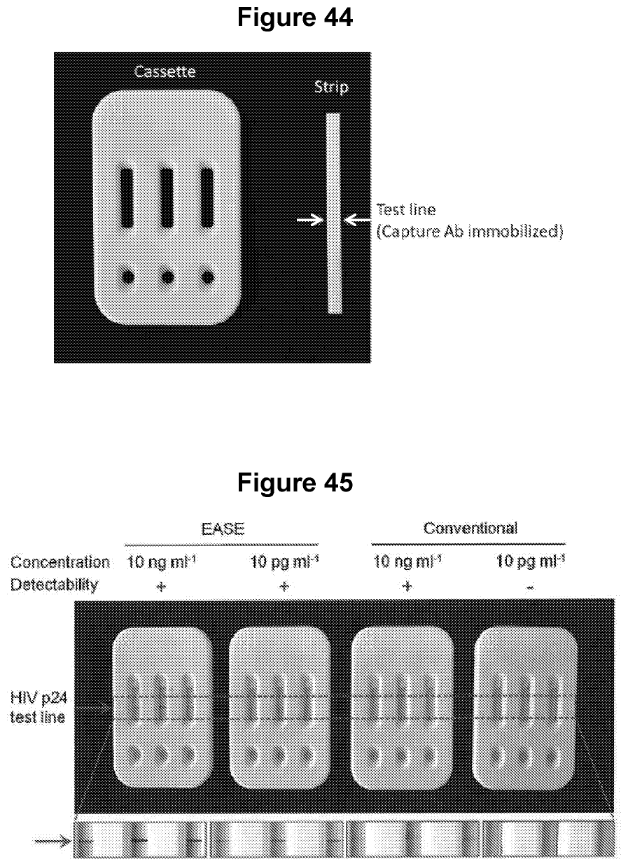

Building on the remarkable sensitivity enhancement achieved on ELISA plates, the HIV biomarker p24 was further tested using lateral flow strips (FIG. 44), a simple and low-cost bioassay, sharing a similar detection mechanism to that of ELISA (conducted in porous membranes rather than on flat surfaces), that is better suited for point-of-care diagnosis.

Lateral flow test-EASE. The striper unit, BioDot ZX010 (BioDot), was equipped with 4 frontline dispensers. Reagents (capture antibody) to be striped were aspirated through the end of the frontline dispenser. The nitrocellulose membrane (Sartorius CN95) was placed on the stage of the striper and secured, and then the frontline dispensers were adjusted to the appropriate position above the nitrocellulose membrane. The striper was programed to release the reagents at a rate of 1 μl cm⁻¹. The membrane was placed in a forced air oven at 37° C. for 30 minutes before cooling in a desiccated environment. Once cooled, the membrane was placed on a backing card (DCN MIBA-020), and then the wick (GE Healthcare, CF5) was laid over the nitrocellulose with a 2 mm overlap. The completed card was placed in the staging area of the guillotine strip cutter (Kinbio ZQ200), and cut into 4 mm wide strips before being stored in Mylar bags that are sealed shut after including desiccant packets until use.

HIV p24 was used as a model analyte for the lateral flow test. Capturing antibodies (HIV p24 antibody) were immobilized onto nitrocellulose membrane, The membrane was blocked with 0.5% tween-20/2% BSA in PBS for 30 minutes. The membrane was then exposed to HIV p24 sample solutions (10 min). After washing (3×), the strips were treated with HIV p24 antibody-HRP conjugates (primary detection reagent) for 30 minutes and washed 3 times again. DAB was used as the enzyme substrate for 10 min color development,

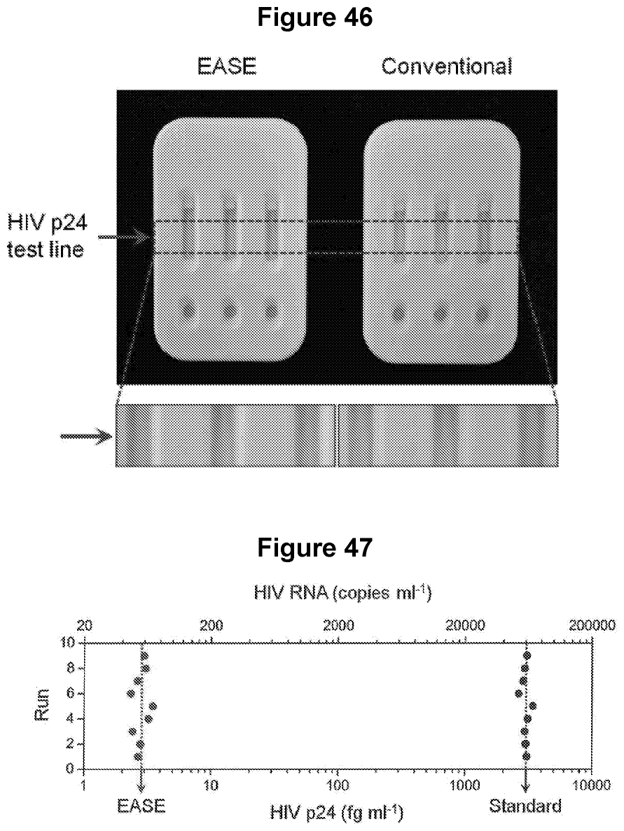

Results. As shown in FIGS. 45-46, the strip test detected p24 at a concentration of 10 ng ml⁻¹ (spiked HIV p24 antigen in phosphate-buffered saline (PBS)) under conventional conditions (using DAB as the substrate), whereas EASE showed improvements of at least 1,000 times that (10 pg ml⁻¹), enabling ultrasensitive detection of HIV antigens with the naked eye,

With the EASE platform validated in the above bioassays, additional biological problems that require much improved detection sensitivity to resolve were addressed. The usefulness of EASE in detection of four biologically significant low-abundance analytes, HIV in blood, in situ protein detection in brain samples, Zika virus (ZIKV) imaging in the placenta, and programmed death-ligand 1 (PD-L1) in tumor, was demonstrated.

### Example 5

### Early Diagnosis of HIV Using ELISA-EASE

Early diagnosis of HIV provides timely access to treatments, thus improving patient outcomes and quality of life. A study of ˜16,000 patients on antiretroviral (ARV) treatments shows substantial numbers of patients beginning ARV later than recommended, due to late diagnosis. For adults, early knowledge of infection also leads to behavioral changes that could reduce 30% of new infections per year. For children and infants, earlier diagnosis is even more important. At this time, over 200,000 children acquire HIV worldwide every year, with most cases due to transmission to infants from their mothers during pregnancy, birth, or breastfeeding. HIV progresses rapidly in infants without treatment they can die within months—but early treatment by ARV greatly improves outcomes, Large-scale programs (e.g., President's Emergency Plan for AIDS Relief (PEPFAR)) have made ARV available, but early diagnosis remains a barrier to treatment.

HIV can be detected in blood or plasma by 1) nucleic acid amplification tests (NAAT), 2) lab based immunoassays (ELISA), or 3) rapid tests (similar to pregnancy tests). In general, NAAT is sensitive, but very expensive, and rapid test is of low performance and cannot be used in infants (false positive due to antibodies from the motherm). For decades, ELISA has been the workhorse laboratory HIV test and is the first test in the Centers for Disease Control and Prevention (CDC) testing algorithm. The sensitivity of ELISA, however, has been a major limitation (even for the most recent generation, detections are made around two weeks after infection). Increasing detection to an earlier time has been a major unmet clinical need.

The ELISA-EASE assay was used to detect p24 antigen, the key protein that makes up most of the viral capsid, in patient sera. Quantitative measurement of its presence in serum is highly valuable to blood screening, diagnosis of infection, and monitoring treatment responses. As recommended by the CDC, HIV p24 antigen detection using ELISA offers a number of advantages such as reduced cost, fast assay times, and applicability in low-resource settings. On the other hand, it is commonly acknowledged that p24 ELISA is an insensitive assay with a LOD of approximately 10 pg ml⁻¹, limiting its use to samples with high viral loads. Incorporating EASE technology, however, can improve the ordinary detection sensitivity of ELISA to extraordinary levels, as shown in the above ELISA studies conducted in buffers.

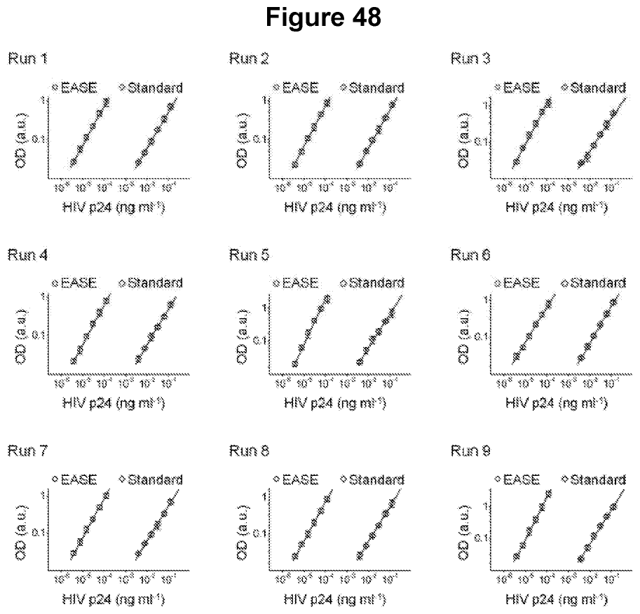

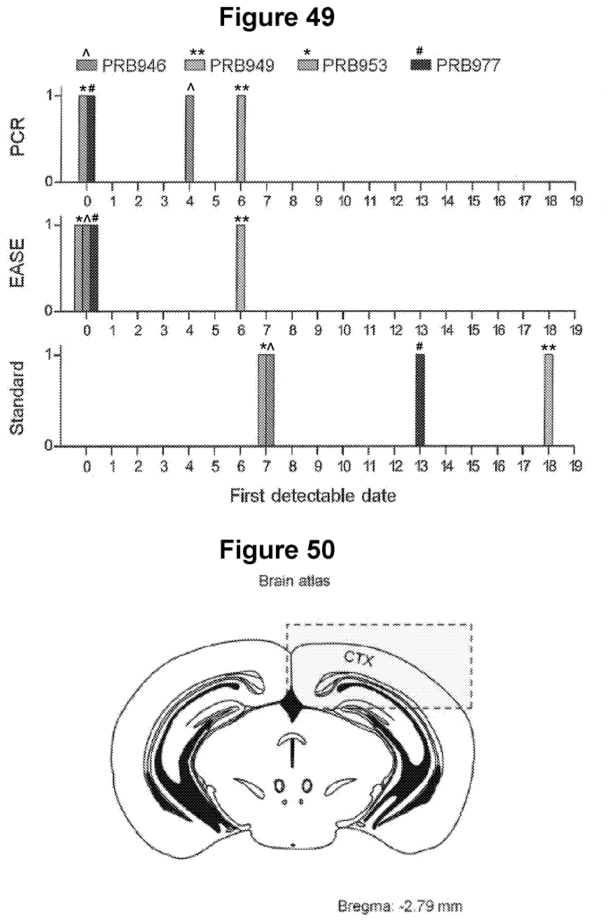

To demonstrate its ability in clinical diagnosis, sera from 24 donors (obtained from SeraCare, Milford, Mass. and Discovery Life Sciences, Los Osos, Calif.) were assayed with either standard ELISA or ELISA with EASE. Among these samples, four were obtained from HIV-infected patients (PRB 946, PRB 949, PRB 953, and PRB 977) whose viral loads had been determined using PCR (data from SeraCare); and 20 HIV-negative donors were included to exclude biased results due to nonspecific interactions (Table 5). The analytical LOD was determined by spiking HIV p24 antigen of various concentrations into plasma. Results from 9 repeated runs performed on 9 consecutive days showed a highly consistent value (FIGS. 47-48; Tables 6-14) of 2.84 fg ml⁻¹ for ELISA-EASE, representing a 1,060-fold improvement over standard ELISA. Theoretical calculations indicate that this level of protein detection corresponds to samples containing approximately 56 copies ml⁻¹ of RNA or 28 ml⁻¹ viral particles, on par with the sensitivity of PCR, which requires sophisticated instruments and long assay time. Indeed, when ELISA-EASE was applied to the HIV infected patient samples (multiple bleeds over a course of 18 days during the development of HIV invention), it could detect the viral infection on average 10 days earlier (similar to FOR) than the standard ELISA assay (Table 15; FIG. 49). This remarkable sensitivity potentially can provide a precious time window for treating other time-sensitive infections (e.g., viral and bacterial infections) and diseases (e.g., heart diseases) as well.

TABLE 5
ELISA-EASE diagnosis of HIV infection in
plasma from healthy blood donors

| Patient ID | EASE-ELISA | Standard ELISA | PCR |
|---|---|---|---|
| R301520 | x | x | x |
| R301522 | x | x | x |
| R301525 | x | x | x |
| R301527 | x | x | x |
| R301537 | x | x | x |
| R301538 | x | x | x |
| R301548 | x | x | x |
| R301549 | x | x | x |
| R301551 | x | x | x |
| R301555 | x | x | x |
| R301556 | x | x | x |
| R301558 | x | x | x |
| R301560 | x | x | x |
| R301563 | x | x | x |
| R301564 | x | x | x |
| R301566 | x | x | x |
| R301571 | x | x | x |
| R301572 | x | x | x |
| R301573 | x | x | x |
| R301574 | x | x | x |

x, below the quantitation range

No positive detection was made using ELISA, ELISA-EASE, or PCR, showing detection specificity across all three methods.

TABLE 6
Run 1

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0273 | 0.0033 | 0.039 | 0.0262 | 0.022 |
| 7.6250E−6 | 0.0568 | 0.1001 | 0.0078 | 0.0461 | 0.0054 |
| 1.5250E−5 | 0.1155 | 0.0175 | 0.0156 | 0.0899 | 0.0130 |
| 3.0500E−5 | 0.2181 | 0.0266 | 0.0313 | 0.1780 | 0.0126 |
| 6.1000E−5 | 0.4825 | 0.0799 | 0.0625 | 0.3395 | 0.0597 |
| 1.2200E−4 | 0.1953 | 0.1953 | 0.1250 | 0.6986 | 0.0993 |

TABLE 7
Run 2

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0232 | 0.0029 | 0.0039 | 0.0241 | 0.0020 |
| 7.6250E−6 | 0.0488 | 0.0066 | 0.0078 | 0.0510 | 0.0052 |
| 1.5250E−5 | 0.1089 | 0.0152 | 0.0156 | 0.0982 | 0.0110 |
| 3.0500E−5 | 0.2099 | 0.0402 | 0.0313 | 0.1830 | 0.0399 |
| 6.1000E−5 | 0.4507 | 0.0523 | 0.0625 | 0.3590 | 0.0300 |
| 1.2200E−4 | 0.8685 | 0.1500 | 0.1250 | 0.7814 | 0.1008 |

TABLE 8
Run 3

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0290 | 0.0035 | 0.0039 | 0.0272 | 0.0021 |
| 7.6250E−6 | 0.0692 | 0.0062 | 0.0078 | 0.0409 | 0.0112 |
| 1.5250E−5 | 0.1589 | 0.0291 | 0.0156 | 0.0812 | 0.0100 |
| 3.0500E−5 | 0.3293 | 0.0507 | 0.0313 | 0.1639 | 0.0151 |
| 6.1000E−5 | 0.6801 | 0.1028 | 0.0625 | 0.3025 | 0.0758 |
| 1.2200E−4 | 1.1912 | 0.2877 | 0.1250 | 0.6343 | 0.0733 |

TABLE 9
Run 4

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0221 | 0.0028 | 0.0039 | 0.0251 | 0.0041 |
| 7.6250E−6 | 0.0430 | 0.0096 | 0.0078 | 0.0466 | 0.0036 |
| 1.5250E−5 | 0.0937 | 0.0126 | 0.0156 | 0.0927 | 0.0200 |
| 3.0500E−5 | 0.1955 | 0.0205 | 0.0313 | 0.1622 | 0.0170 |
| 6.1000E−5 | 0.3956 | 0.0777 | 0.0625 | 0.3023 | 0.0353 |
| 1.2200E−4 | 0.7755 | 0.1159 | 0.1250 | 0.6309 | 0.0955 |

TABLE 10
Run 5

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0209 | 0.0038 | 0.0039 | 0.0231 | 0.0033 |
| 7.6250E−6 | 0.0639 | 0.0103 | 0.0078 | 0.0520 | 0.0111 |
| 1.5250E−5 | 0.1745 | 0.0488 | 0.0156 | 0.1123 | 0.0286 |
| 3.0500E−5 | 0.4099 | 0.0590 | 0.0313 | 0.1921 | 0.0380 |
| 6.1000E−5 | 0.9633 | 0.1022 | 0.0625 | 0.3924 | 0.0403 |
| 1.2200E−4 | 1.8988 | 0.4633 | 0.1250 | 0.7233 | 0.1995 |

TABLE 11
Run 6

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0311 | 0.0057 | 0.0039 | 0.0278 | 0.0025 |
| 7.6250E−6 | 0.0524 | 0.0068 | 0.0078 | 0.0564 | 0.0091 |
| 1.5250E−5 | 0.1052 | 0.0166 | 0.0156 | 0.1086 | 0.0106 |
| 3.0500E−5 | 0.2154 | 0.0177 | 0.0313 | 0.2130 | 0.0124 |
| 6.1000E−5 | 0.4249 | 0.0649 | 0.0625 | 0.4360 | 0.0576 |
| 1.2200E−4 | 0.7890 | 0.1781 | 0.1250 | 0.8801 | 0.1031 |

TABLE 12
Run 7

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0290 | 0.0039 | 0.0039 | 0.0284 | 0.0027 |
| 7.6250E−6 | 0.0584 | 0.0111 | 0.0078 | 0.0522 | 0.0039 |
| 1.5250E−5 | 0.1276 | 0.0233 | 0.0156 | 0.0920 | 0.0110 |
| 3.0500E−5 | 0.2388 | 0.0209 | 0.0313 | 0.1651 | 0.0390 |
| 6.1000E−5 | 0.4967 | 0.0757 | 0.0625 | 0.3299 | 0.0373 |
| 1.2200E−4 | 0.9999 | 0.1604 | 0.1250 | 0.6789 | 0.1002 |

TABLE 13
Run 8

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0248 | 0.0035 | 0.0039 | 0.0260 | 0.0056 |
| 7.6250E−6 | 0.0526 | 0.0071 | 0.0078 | 0.0480 | 0.0044 |
| 1.5250E−5 | 0.1012 | 0.0195 | 0.0156 | 0.0897 | 0.0104 |
| 3.0500E−5 | 0.2097 | 0.0396 | 0.0313 | 0.1744 | 0.0151 |
| 6.1000E−5 | 0.4355 | 0.0552 | 0.0625 | 0.3638 | 0.0598 |
| 1.2200E−4 | 0.9240 | 0.1559 | 0.1250 | 0.6987 | 0.1594 |

TABLE 14
Run 9

| EASE | | | | | |
|---|---|---|---|---|---|
| HIV p24 | | | Conventional | | |
| (ng ml⁻¹) | OD | S.D. | (ng ml⁻¹) | OD | S.D. |
| 3.8125E−6 | 0.0276 | 0.0038 | 0.0039 | 0.0233 | 0.0023 |
| 7.6250E−6 | 0.0604 | 0.0099 | 0.0078 | 0.0515 | 0.0099 |
| 1.5250E−5 | 0.1724 | 0.0455 | 0.0156 | 0.1206 | 0.0236 |
| 3.0500E−5 | 0.3998 | 0.0784 | 0.0313 | 0.2502 | 0.0269 |
| 6.1000E−5 | 0.9756 | 0.2248 | 0.0625 | 0.4995 | 0.0403 |
| 1.2200E−4 | 2.5063 | 0.4858 | 0.1250 | 0.9753 | 0.1596 |

TABLE 15
Viral load assessmenent using ELISA,
ELISA-EASE, and PCR in four HIV-
infected patients' plasma samples.

| Patient | Phlebotomy | EASE | EASE | Standard | Standard |
|---|---|---|---|---|---|
| ID | Date (days) | (pg ml⁻¹) | CV (%) | (pg ml⁻¹) | CV (%) |
| PRB | 0 | 0.006* | 15.8 | x | N/A |
| 946 | 4^{†} | 0.807 | 9.96 | x | N/A |
| | 7 | 26.86 | 10.2 | 19.22* | 8.48 |
| | 11 | 39.70 | 17.5 | 50.63 | 10.1 |
| PRB | 0 | x | N/A | x | N/A |
| 949 | 6^{†} | 0.029 | 13.6 | x | N/A |
| | 9 | 0.561 | 9.48 | x | N/A |
| | 18 | 22.05 | 18.1 | 17.22* | 10.9 |
| PRB | 0^{†} | 0.043* | 11.2 | x | N/A |
| 953 | 3 | 1.320 | 17.3 | x | N/A |
| | 7 | 23.36 | 8.79 | 16.01* | 7.39 |
| | 10 | 39.99 | 18.8 | 50.97 | 15.7 |
| PRB | 0^{†} | 0.009* | 12.0 | x | N/A |
| 977 | 2 | 0.121 | 7.65 | x | N/A |
| | 13 | >100 | N/A | >100* | N/A |
| | 15 | >100 | N/A | >100 | N/A |

x, below the quantitation range; ^{†}, first detectable date using PCR; *, first detectable date using ELISA. Measurement variabilities were calculated based on coefficient of variation (CV), which was lower than 20% in all measurements.

### Example 6

### Resolving Corticotrophin Eeleasing Factor (CRF) Distribution in the Brain Using IF-EASE

CRF and its canonical G-protein coupled receptors, corticotrophin releasing factor receptor type 1 (CRFR1) and CRFR2 play an essential role in stress responsiveness regulated by the central nervous system. Alterations in the function of the CRF system and changes in CRF receptor signaling are broadly linked to neuropsychiatric disorders including addiction and depression. The ability to resolve the spatial distribution of CRF receptors in the brain will transform our understanding of how these receptors influence neural circuit function and how alterations in the expression and distribution of these receptors contribute to the disease states. Detection of CRF receptors has been largely limited to in situ hybridization detection on the mRNA level and radio-ligand binding assays, which provide poor spatial resolution. High-resolution localization of these receptors using conventional immunostaining techniques has been limited by the low levels of receptor expression. To test the effectiveness of EASE technology to enhance CRFR1 detection using antibody staining, immunostaining for CRFR1 was performed using conventional methods and EASE.

Histology preparation of brain tissues for CRFR1 staining. Mice were deeply anesthetized with 50 mg/kg of Beuthanasia-D and transcardially perfused with phosphate-buffered saline (PBS), followed by 4% paraformaldehyde. Whole brain tissue was dissected, fixed overnight in 4% paraformaldehyde, and cryoprotected by soaking in a 30% sucrose solution for 48 hours. The brains were flash frozen in OCT and stored at −80° C. The frozen brains were then cryosectioned to 30 μm-thick sections and stored in lx PBS with 0.1% NaAz prior to immunostaining.

CRFR1 IF staining in brain sections. Coronal 30 μm sections were selected based on a reference atlas (Franklin and Paxinos) and analyzed for protein expression. Primary antibody against CRFR1 (Nevus Biologicals, cat. No. NLS1778) (intermediate detection reagent) was diluted 1:100. Cy3- or HRP-labeled secondary antibodies (donkey anti-rabbit, Jackson Immunolabs, and goat anti-rabbit) (conventional reagent or primary detection reagent, respectively) were diluted 1:250. Sections were incubated in 3% hydrogen peroxide 1×TBS buffer (10 min) to quench the intrinsic peroxide in tissue, washed with 1× TBS for 10 minutes, and blocked with 1× TBST (TBS+0.3% TritonX 100) with 3% donkey serum for 60 minutes. The blocked sections were stained with the primary antibody diluted in the blocking buffer overnight, washed three times in lx TBS for 10 minutes, and incubated in Cy3- or HRP-conjugated secondary antibodies for 1 hour at room temperature. IF-EASE was applied as described in the Examples above (amine-Cy3, a secondary detection reagent, was used as the reactive fluorophore). The sections were washed three more times in 1× TBS and mounted.

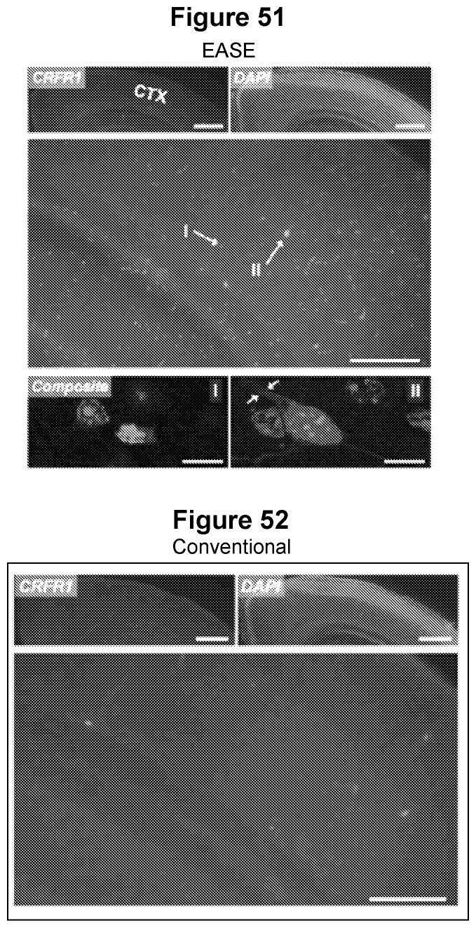

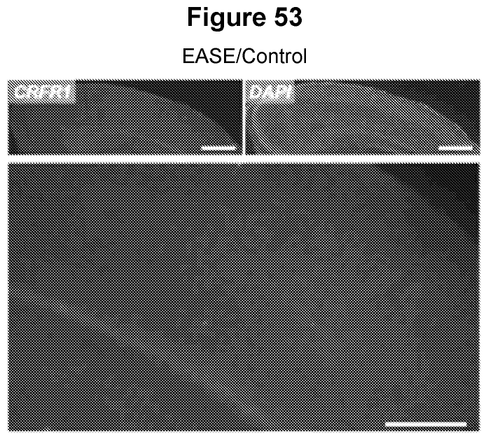

Results. Analysis of CRFR1 detection revealed only a small number of CRFR1-positive cells in the cerebral cortex of the mouse brain using conventional immunostaining (FIGS. 50-53). In contrast, EASE amplification revealed numerous CRFR1-positive cells including both small diameter and large diameter cells, indicative of expression in both interneurons and pyramidal neurons, respectively (FIG. 51). Additionally, EASE detection of CRFR1 localized the protein to the cell bodies of both cell types, as well as the apical dendrites of pyramidal neurons.

### Example 7

### Direct Imaging of ZIKV Infection in the Placenta Using IF-EASE

Zika is a mosquito-borne flavivirus initially identified in the 1950s' in monkeys. Its recent outbreak in Brazil has been correlated with cases of fetal microcephaly as well as Guillian Barré, raising major global concerns. While there is now scientific consensus, including our own work, that ZIKV indeed causes fatal brain injury, the mechanism of how it occurs is largely unknown. qPCR and deep sequencing are capable of identifying ZIKV in the placenta, but cannot elucidate the means by which ZIKV crossed the placental barrier due to their inability to track ZIKV through conventional immunohistologic analysis.

Immunostaining of ZIKV-infected placenta. Placental samples were collected from pregnant pigtail macaques (Macaca nemestrina), who were inoculated with ZIKV (strain FSS13025, Cambodia 2010) or from a normal pregnancy. Formaldehyde-fixed sections of frozen placental chorionic villi were stained using both conventional IF and IF-EASE. The primary antibody (ZIKV E-protein Clone ZV-13, Diamond lab) (intermediate detection reagent) was diluted 1:200. Other reagents such as the primary detection reagent as well as the staining protocol were the same as that described in the CRFR1 experiments. A healthy control was used for studying the specificity of IF-EASE. Adjacent tissue slides were used for all staining conditions.

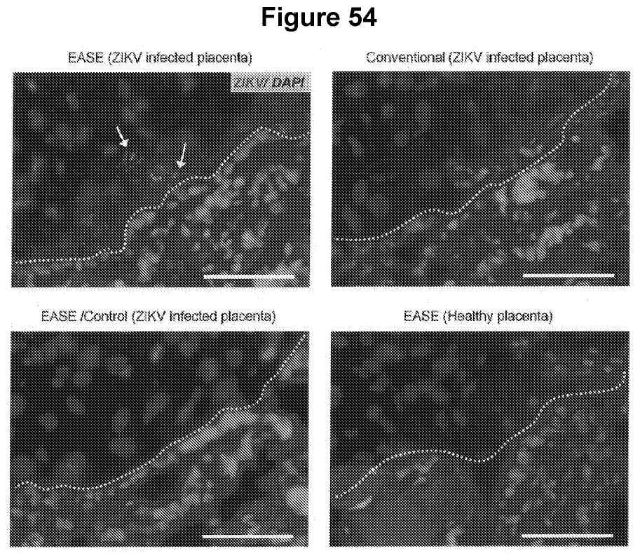

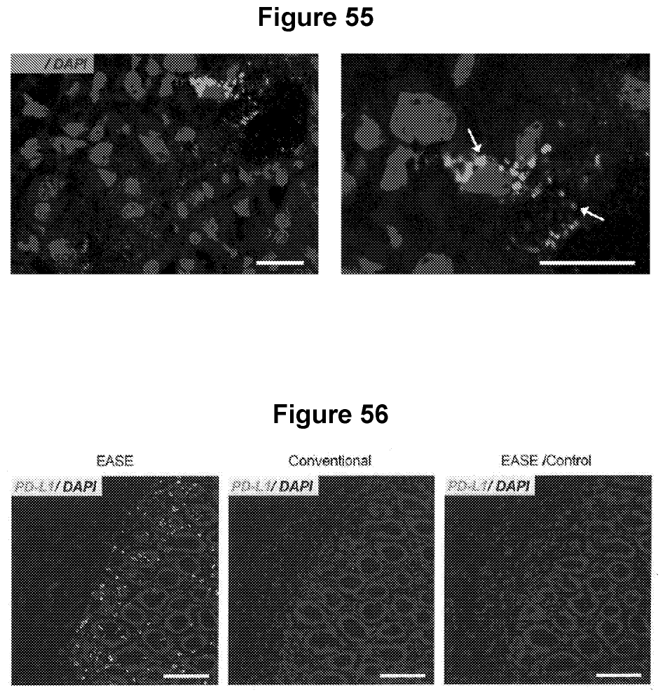

Results. The EASE technology enabled direct visualization of ZIKV-infected cells within the placental chorionic villus core of pregnant nonhuman primates. As shown in FIGS. 54-55, the infected cells appeared in the mesenchymal core in close proximity to the cytotrophoblast cell layer. The EASE technology opens a new avenue to understand fetal brain injury and microcephaly caused by ZIKV and potentially to prevent mother-to-child transmission.

### Example 8

### PD-L1 Imaging in Patient Tumor Specimens Using IF-EASE

PD-L1 also known as CD-274 or B7-H1, is a cell surface ligand, which binds and triggers PD-1, a potent immune-inhibitory receptor on T cells49. Monoclonal antibodies which block this interaction, by binding either PD-L1 or PD-1, have proven to be efficacious immune-oncology agents in a variety of tumor types. Immunohistochemical assays for detecting PD-L1+ cells within tumors have also been approved as companion diagnostic tests for patient selection in limited therapeutic indications, but broader application of anti-PDL1 IHC is limited by both biologic and technical factors. PD-L1 expression vary broadly across a wide range and levels below the detection thresholds of current IHC assays still have biologic significance. Therefore, it was determined whether EASE can be used to detect low-level PD-L1 signals while preserving good signal-to-noise ratios, an unmet clinical need for immunotherapy. Clinical formalin-fixed paraffin-embedded (FFPE) pancreatic tumor specimens with low PD-L1 expression were used to test the performance of IF-EASE with conventional IF.

PD-L1 immunostaining of pancreatic tumor specimens. The FFPE pancreatic tumor tissue specimens from two patients (SU-09-21157; SU-10-26808) were deparaffinized by washing the slides with xylene (7 min, 3 times), 100% ethanol (2 min, twice), 95% ethanol (2 min, twice), 70% ethanol (2 min, twice) and DI water (2 min). The sections were then incubated in 3% hydrogen peroxide in 1× TBS buffer (30 min) to quench the intrinsic peroxide. Antigen retrieval was performed by incubating the sections with the Trilogy antigen retrieval buffer under high pressure (15 min), cooling down (20 min), and washing with ix TBS (5 min, 2 times). The sections were subsequently stained using both conventional IF and IF-EASE. The protocols are the same as the ones described immediately above, except the primary antibody (intermediate detection reagent) is mouse anti-PD-L1 (1:150 dilution, Cell signaling Technology, REF: 29122S). Adjacent tissue slides were used for all staining conditions.

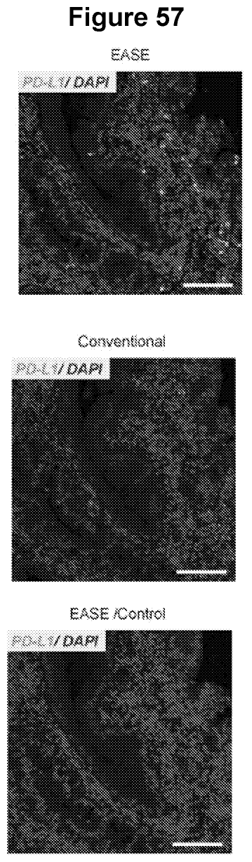

Results. As shown in FIGS. 56-57, specific detection of PD-L1 was readily achieved with IF-EASE, whereas the signals detected by conventional IF technology were at extremely low levels. These exciting results address the unmet clinical need of detecting low abundance analytes in FFPE tissues (high autofluorescence background).

HRP can speed up PDA polymerization by approximately 300 times. More importantly, due to the excellent reactivity of PDA to primary amines, the polymer chains quickly crosslink with nearby biomolecules (rich in many reactive chemical groups including NH2), forming a localized network for immobilization of a large number of reporter molecules and nanoparticles (having accessible amine groups) for signal enhancement, while preserving the spatial information. This technology, dubbed EASE, is useful in a number of contexts including immunohistochemistry and immunofluorescence for single cell imaging, ELISA, lateral flow strips, and suspension microarrays, as highlighted below in Table 16, summarizing the assays of Examples 2-8. Consistently, it improves bio-imaging and—detection sensitivity by at least 2-3 orders of magnitude, regardless of the assay format. Most significantly, EASE achieves this remarkable sensitivity without changing the design of common assay formats, or requiring specialized equipment and reagents, in contrast to most ultrasensitive detection technologies invented in the past 10-20 years. Therefore, EASE can be directly incorporated into the current biological and clinical infrastructure for immediate impact.

TABLE 16
Assay Formats of Examples 2-8.

| Assay | Format | |
|---|---|---|
| Immunohisto- | Primary | Rabbit anti-Lamin A |
| chemistry (IHC)/ | antibody | (HSP90, Ki67, |
| Immuno- | (1′Ab) | Cox-4 or GAPDH) IgG |
| fluorescence (IF) | Secondary | EASE: 2′ Ab-HRP (IHC and IF) |
| | antibody | Conventional: 2′ Ab-HRP (IHC); |
| | (2′Ab) | 2′Ab-QD (IF) |
| | Signal | EASE: EASE substrate (IHC); |
| | development | EASE substrate/QD-NH₂ (IF) |
| | | Conventional: DAB substrate |
| | | (IHC); 2′Ab-QD (IF) |
| Suspension | Bead | Coated with IgG (mouse or |
| microarray | | rabbit) |
| | Analyte | Biotinylated 2′Ab |
| | Signal | EASE: Strepdavidin-HRP/ |
| | development | EASE substrate/QD-NH₂ |
| | | Conventional: Strepdavidin-QD |
| Enzyme-linked | First layer of | Capture Ab |
| immunosorbent | sandwich | |
| assay (ELISA) | Second layer | Analyte (Mouse IgG, HIV |
| | of sandwich | p24, KLK2, CRP or |
| | | VEGF) |
| | Third layer of | Detection Ab-HRP |
| | sandwich | |
| | Signal | EASE: EASE substrate/HRP/ |
| | development | TMB substrate |
| | | Conventional: TMB substrate |
| Lateral flow test | First layer of | Capture Ab |
| | sandwich | |
| | Second Layer | Analyte (HIV p24) |
| | of sandwich | |
| | Third layer of | Detection Ab-HRP |
| | sandwich | |
| | Signal | EASE: EASE substrate/HRP/ |
| | development | DAB substrate |
| | | Conventional: DAB substrate |

The flexibility of this general technology has been demonstrated to be useful in a number of real biological problems that cannot be solved (or are at least extremely difficult to solve) using conventional bioassays. EASE was applied to ELISA-based detection of HIV infection in patient blood samples. For comparison, the measurements were benchmarked against the gold-standard assays, standard ELISA and PCR. The EASE-enabled ELISA outperformed the standard ELISA by >1,000 times in sensitivity, which translates into detection of 2-3 viruses per 100 μl of blood. This sensitivity is similar to that of PCR-based approaches allowing HIV detection 1-2 weeks earlier, yet ELISA is faster and cheaper to perform, and compatible with point-of-care (POC) applications (rending equipment such as a costly thermocycler unnecessary). Furthermore, EASE is a robust process that can be applied to a variety of real biological and clinical problems, such as brain biology, in situ virus imaging in placenta, and PD-L1 imaging for immunotherapy.

### REFERENCES

Howes, P. D., Chandrawati, R. & Stevens, M. M. Colloidal nanoparticles as advanced biological sensors. Science 346, 1247390 (2014).
Chan, W. C. & Nie, S. Quantum dot bioconjugates for ultrasensitive nonisotopic detection. Science 281, 2016-2018 (1998).
Kelley, S. O. et al. Advancing the speed, sensitivity and accuracy of biomolecular detection using multi-length-scale engineering. _u Nature nanotechnology 9, 969-980 (2014).
Nam, J.-M., Thaxton, C. S. & Mirkin, C. A. Nanoparticle-based bio-bar codes for the ultrasensitive detection of proteins. Science 301, 1884-1886 (2003).
Kosaka, P. M. et al. Detection of cancer biomarkers in serum using a hybrid mechanical and optoplasmonic nanosensor. Nature nanotechnology 9, 1047-1053 (2014).
Rodriguez-Lorenzo, L., de La Rica, R., Alvarez-Puebla, R. A., Liz-Marzan, L. M. & Stevens, M. M. Plasmonic nanosensors with inverse sensitivity by means of enzyme-guided crystal growth. Nature materials 11, 604-607 (2012).
He, L., Ozdemir, S. K., Zhu, J., Kim, W. & Yang, L. Detecting single viruses and nanoparticles using whispering gallery microlasers. Nature nanotechnology 6, 428-432 (2011).
Thomas, R. K. et al. Sensitive mutation detection in heterogeneous cancer specimens by massively parallel picoliter reactor sequencing. Nature Medicine 12, 852-855 (2006).
Zheng, G., Patolsky, F., Cui, Y., Wang, W. U. & Lieber, C. M. Multiplexed electrical detection of cancer markers with nanowire sensor arrays. Nature Biotechnology 23, 1294-1301 (2005).
Wu, G. et al. Bioassay of prostate-specific antigen (PSA) using microcantilevers. Nature Biotechnology 19, 856-860 (2001).
Schallmeiner, E. et al. Sensitive protein detection via triple-binder proximity ligation assays. Nature Methods 4, 135-137 (2007).
Watanabe, R. et al. Arrayed lipid bilayer chambers allow single-molecule analysis of membrane transporter activity. Nature communications 5 (2014).
Ma, W. et al. Attomolar DNA detection with chiral nanorod assemblies. Nature communications 4 (2013).
Haun, J. B., Devaraj, N. K., Hilderbrand, S. A., Lee, H. & Weissleder, R. Bioorthogonal chemistry amplifies nanoparticle binding and enhances the sensitivity of cell detection. Nature nanotechnology 5, 660-665 (2010)
Vollmer, F. & Arnold, S. Whispering-gallery-mode biosensing: label-free detection down to single molecules. Nature Methods 5, 591-596 (2008).
Li, M., Tang, H. X. & Roukes, M. L. Ultra-sensitive NEMS-based cantilevers for sensing, scanned probe and very high-frequency applications. Nature nanotechnology 2, 114-120 (2007).
Cooper, M. A. et al. Direct and sensitive detection of a human virus by rupture event scanning. Nature Biotechnology 19, 833-837 (2001).
Rissin, D. M. et al. Single-molecule enzyme-linked immunosorbent assay detects serum proteins at subfemtomolar concentrations. Nature Biotechnology 28, 595-599 (2010).
Burst bubbles. Nature 526, 609-610 (2015).
Lee, H., Dellatore, S. M., Miller, W. M. & Messersmith, P. B. Mussel-inspired surface chemistry for multifunctional coatings. Science 318, 426-430 (2007).
Lee, H., Rho, J. & Messersmith, P. B. Facile conjugation of biomolecules onto surfaces via mussel adhesive protein inspired coatings. Advanced Materials 21, 431-434 (2009).
Soderhall, K. & Cerenius, L. Role of the prophenoloxidase-activating system in invertebrate immunity. Current Opinion in Immunology 10, 23-28 (1998).
Cerenius, L. & Soderhall, K. The prophenoloxidase-activating system in invertebrates. Immunological Reviews 198, 116-126 (2004).
Weber, R. et al. Threshold of detection of Cryptosporidium oocysts in human stool specimens: evidence for low sensitivity of current diagnostic methods. Journal of Clinical Microbiology 29, 1323-1327 (1991).
Mahler, M., Ngo, J. T., Schulte-Pelkum, J., Luettich, T. & Fritzler, M. J. Limited reliability of the indirect immunofluorescence technique for the detection of anti-Rib-P antibodies. Arthritis Research & Therapy 10, 1 (2008).
Zrazhevskiy, P., Sena, M. & Gao, X. Designing multifunctional quantum dots for bioimaging, detection, and drug delivery. Chemical Society Reviews 39, 4326-4354 (2010).
Medintz, I. L., Uyeda, H. T., Goldman, E. R. & Mattoussi, H. Quantum dot bioconjugates for imaging, labelling and sensing. Nature materials 4, 435-446 (2005).
Zrazhevskiy, P. et al. Cross-platform DNA encoding for single-cell imaging of gene expression. Angewandte Chemie International Edition 55, 8975-8978 (2016).
Battersby, B. J., Lawrie, G. A. & Trau, M. Optical encoding of microbeads for gene screening: alternatives to microarrays. Drug Discovery Today 6, 19-26 (2001).
Braeckmans, K., De Smedt, S. C., Leblans, M., Pauwels, R. & Demeester, J. Encoding microcarriers: present and future technologies. Nature Reviews Drug Discovery 1, 447-456 (2002)
Nolan, J. P. & Sklar, L. A. Suspension array technology: evolution of the flat-array paradigm. TRENDS in Biotechnology 20, 9-12 (2002).
Fulton, R. J., McDade, R. L., Smith, P. L., Kienker, L. J. & Kettman, J. R. Advanced multiplexed analysis with the FlowMetrix™ system. Clinical chemistry 43, 1749-1756 (1997).
Dunbar, S. A. Applications of Luminex xMAP technology for rapid, high-throughput multiplexed nucleic acid detection. Clinica Chimica Acta 363, 71-82 (2006).
Chan, W. C. et al. Luminescent quantum dots for multiplexed biological detection and imaging. Current Opinion in Biotechnology 13, 40-46 (2002).
Han, M., Gao, X., Su, J. Z. & Nie, S. Quantum-dot-tagged microbeads for multiplexed optical coding of biomolecules. Nature Biotechnology 19, 631-635 (2001).
Klein, D., Hurley, L. B., Merrill, D. & Quesenberry Jr, C. P. Review of medical encounters in the 5 years before a diagnosis of HIV-1 infection: implications for early detection. JAIDS Journal of Acquired Immune Deficiency Syndromes 32, 143-152 (2003).
Fu, E. et al. Enhanced sensitivity of lateral flow tests using a two-dimensional paper network format. Analytical chemistry 83, 7941-7946 (2011).
Palella, F. J. et al. Survival benefit of initiating antiretroviral therapy in HIV-infected persons in different CD4+ cell strata. Annals of internal medicine, 138, 620-626 (2003).
Holodniy, M. et al. Relationship between antiretroviral prescribing patterns and treatment guidelines in treatment-naive HIV-1-infected US veterans (1992-2004). Journal of Acquired Immune Deficiency Syndromes, 44, 20-29 (2007).
Marks, G., Crepaz, N. & Janssen, R. S. Estimating sexual transmission of HIV from persons aware and unaware that they are infected with the virus in the USA. Aids, 20, 1447-1450 (2006).
Miles, S. A. et al. Rapid serologic testing with immune-complex-dissociated HIV p24 antigen for early detection of HIV infection in neonates. New England Journal of Medicine 328, 297-302 (1993).
Nishanian, P., Huskins, K. R., Stehn, S., Detels, R. & Fahey, J. L. A simple method for improved assay demonstrates that HIV p24 antigen is present as immune complexes in most sera from HIV-infected individuals. Journal of Infectious Diseases 162, 21-28 (1990).
Marozsan, A. J. et al. Relationships between infectious titer, capsid protein levels, and reverse transcriptase activities of diverse human immunodeficiency virus type 1 isolates. Journal of Virology 78, 11130-11141 (2004).
Bale, T. L. & Vale, W. W. CRF and CRF receptors: role in stress responsivity and other behaviors. Annual Review of Pharmacology and Toxicology, 44, 525-557 (2004)
Zorrilla, E. P., Logrip, M. L., & Koob, G. Corticotropin releasing factor: a key role in the neurobiology of addiction. Frontiers in neuroendocrinology, 35, 234-244 (2014).
Van Pett, K. et al. Distribution of mRNAs encoding CRF receptors in brain and pituitary of rat and mouse. Journal of Comparative Neurology, 428, 191-212 (2000).
Weathington, J. M. & Cooke, B. M. Corticotropin-releasing factor receptor binding in the amygdala changes across puberty in a sex-specific manner. Endocrinology, 153, 5701-5705 (2012).
Waldorf, K. M. A. et al. Fetal brain lesions after subcutaneous inoculation of Zika virus in a pregnant nonhuman primate. Nature Medicine, 22, 1256-1259 (2016).
Keir, M. E. et al. PD-1 and its ligands in tolerance and immunity. Annual Review of Immunology, 26, 677-704 (2008).
Brahmer, J. R. et al. Phase I study of single-agent anti-programmed death-1 (MDX-1106) in refractory solid tumors: safety, clinical activity, pharmacodynamics, and immunologic correlates. Journal of Clinical Oncology, 28, 3167-3175 (2010).
Hamid, O. et al. Safety and tumor responses with lambrolizumab (anti-PD-1) in melanoma. New England Journal of Medicine, 369, 134-144 (2013).
Garon, E. B. et al. Pembrolizumab for the treatment of non-small-cell lung cancer. New England Journal of Medicine, 372, 2018-2028 (2015).
Rizvi, N. A. et al. Activity and safety of nivolumab, an anti-PD-1 immune checkpoint inhibitor, for patients with advanced, refractory squamous non-small-cell lung cancer (CheckMate 063): a phase 2, single-arm trial. The Lancet Oncology, 16, 257-265 (2015).
Nghiem, P. T. et al. PD-1 blockade with pembrolizumab in advanced Merkel-cell carcinoma. New England Journal of Medicine, 374, 2542-2552 (2016).
Wang, X., et al. PD-L1 expression in human cancers and its association with clinical outcomes. OncoTargets and Therapy, 9, 5023-5039 (2016).
Liu, Y., Ai, K. & Lu, L. Polydopamine and its derivative materials: synthesis and promising applications in energy, environmental, and biomedical fields. Chemical Reviews 114, 5057-5115 (2014).
Li, J. et al. Stably doped conducting polymer nanoshells by surface initiated polymerization. Nano Letters 15, 8217-8222 (2015).
Sanford, C. A., et al. A central amygdala CRF circuit facilitates learning about weak threats. Neuron 93, 164-178 (2017).
Zhao, H. et al, Structural basis of Zika virus-specific antibody protection, Cell 166, 1016-1027 (2016)

1. A method for polymerizing a polyphenol, comprising:
providing a polyphenol;
providing an enzyme having peroxidase-like activity;
contacting the polyphenol and an oxidant with the enzyme having peroxidase-like activity, under conditions sufficient to polymerize the polyphenol to form a polyphenol polymer.

2. A method according to claim 1, wherein the polyphenol polymer forms as a precipitate.

3. A method according to claim 2, wherein the polyphenol polymer forms a surface coating on a surface.

4. A method according to claim 1, wherein the enzyme having peroxidase activity is not immobilized at a surface.

5. A method according to claim 1, wherein the enzyme having peroxidase-like activity is in aqueous solution or suspension when it is contacted with the polyphenol and the oxidant.

6. A method for depositing a polyphenol polymer on a surface, comprising
providing, at a target site, an enzyme having peroxidase-like activity immobilized at the surface; and
polymerizing, at the target site, a polyphenol in the presence of an oxidant and the enzyme to provide the polyphenol polymer, deposited on the surface.

7. A method according to claim 6, wherein the enzyme is adsorbed onto the surface, or wherein the enzyme is linked to the surface via a streptavidin-biotin interaction, or wherein the enzyme is linked to the surface via an antibody-antigen interaction, or wherein the enzyme is linked to the surface via a silane coupling agent.

8.-10. (canceled)

11. A method for detecting an analyte comprising
providing
a sample comprising the analyte; and
a primary detection reagent, linked to an enzyme having peroxidase-like activity;
incubating the sample in the presence of the primary detection reagent to provide a target site comprising a complex of the analyte and the detection reagent;
polymerizing, at the target site, a polyphenol in the presence of an oxidant and the enzyme to provide a polyphenol polymer; and
detecting the presence of polyphenol polymer.

12. A method according to claim 11, wherein the primary detection reagent comprises an antibody, a peptide, an oligonucleotide, or their derivatives, or wherein the primary detection reagent comprises streptavidin.

13. (canceled)

14. A method according to claim 11, wherein the primary detection reagent is capable of binding the analyte.

15. A method according to claim 11, further comprising providing an intermediate detection reagent capable of binding the analyte, wherein
the primary detection reagent is capable of binding the intermediate detection reagent; and
incubation is further in the presence of the intermediate detection reagent, to provide a target site comprising a complex of the analyte, intermediate detection reagent, and primary detection reagent.

16. A method according to claim 15, wherein the intermediate detection reagent comprises an antibody, or wherein the intermediate detection reagent comprises a biotin-labeled affinity molecule.

17.-29. (canceled)

30. A method according to claim 11, comprising
providing
a sample comprising the analyte, the analyte immobilized on a cell surface or localized in a cell compartment;
an intermediate detection reagent capable of binding the analyte; and
a primary detection reagent linked to an enzyme having peroxidase-like activity, the primary detection reagent capable of binding the intermediate detection reagent;
incubating the sample in the presence of the intermediate detection reagent, to provide a target site comprising a complex of the analyte and intermediate detection reagent;
incubating the sample in the presence of the primary detection reagent, to provide a target site comprising a complex of the analyte, the intermediate detection reagent, and the primary detection reagent;
polymerizing, at the target site, a polyphenol in the presence of an oxidant to provide a polyphenol polymer; and
detecting the presence of polyphenol polymer

31.-32. (canceled)

33. A method according to claim 11, comprising
providing
a sample comprising the analyte, the analyte bound to a capture reagent, the capture reagent immobilized on a microsphere; and
a primary detection reagent linked to an enzyme having peroxidase-like activity, the primary detection reagent capable of binding the analyte;
incubating the sample in the presence of the primary detection reagent, to provide a target site comprising a complex of the analyte and the primary detection reagent;
polymerizing, at the target site, a polyphenol derivative in the presence of an oxidant to provide a polyphenol polymer;
incubating the polyphenol polymer in the presence of a secondary detection reagent comprising an amine-functionalized tag; and
detecting the presence of polyphenol polymer, wherein detection comprises measuring the absorption or emission of the secondary detection agent.

34. A method according to claim 11, comprising
providing
a sample comprising the analyte, the analyte bound to a capture reagent, the capture reagent immobilized on a solid support; and
a primary detection reagent linked to an enzyme having peroxidase-like activity, the primary detection reagent capable of binding the analyte;
incubating the sample in the presence of the primary detection reagent, to provide a target site comprising a complex of the analyte and the primary detection reagent;
polymerizing, at the target site, a polyphenol in the presence of an oxidant to provide a polyphenol polymer;
incubating the polyphenol polymerin the presence of a secondary detection agent comprising an enzyme capable of catalyzing the conversion of a chromogenic substrate; and
detecting the presence of polyphenol polymer, wherein detection comprises measuring the absorption or emission of the chromogenic substrate.

35.-43. (canceled)

44. A method according to claim 1, wherein the polyphenol is selected from the group consisting of elegeic acid, theaflavin-3-gallage, gallic acid, tannic acid, pyrogallol, catechol, catechin, epigallocatechin, epigallocatechin, quercetin, morin, naringenin, rutin, naringin, phloroglucinol, hydroquinone, resorcinol, hydroxyhydroquinone, resveratrol, dopamine, -and derivatives thereof.

45. A method according to claim 1, wherein the polyphenol has a molecular weight of no more than 1000 g/mol, e.g., no more than 800 g/mol or even no more than 500 g/mol.

46. A method according to claim 1, wherein the polyphenol polymer is a polydopamine, e.g., a polymer of a dopamine derivative or a polymer of dopamine.

47. A method according to claim 46, wherein the polydopamine is a copolymer of dopamine and/or a dopamine derivative with another polyphenol.

48. A method according to claim 1, wherein the enzyme comprises a polypeptide, a ribozyme, or a deoxyribozyme.

49.-69. (canceled)
